(19) United States
(12) Patent Application Publication
(10) Pub. No.: US 2024/0105277 A1
(43) Pub. Date: Mar. 28, 2024

(54) DRUG DISCOVERY VIA REINFORCEMENT LEARNING WITH THREE-DIMENSIONAL MODELING

(71) Applicant: BATTELLE MEMORIAL INSTITUTE, Richland, WA (US)

(72) Inventors: Neeraj Kumar, Richland, WA (US); Mridula V.S. Bontha, Austin, TX (US); Andrew D. McNaughton, Richland, WA (US); Carter R. Knutson, Salem, OR (US); Jenna A. Pope, Richland, WA (US)

(73) Assignee: BATTELLE MEMORIAL INSTITUTE, Richland, WA (US)

(21) Appl. No.: 18/370,814

(22) Filed: Sep. 20, 2023

Related U.S. Application Data
(60) Provisional application No. 63/461,531, filed on Apr. 24, 2023.
(60) Provisional application No. 63/409,006, filed on Sep. 22, 2022.

(51) Int. Cl. G16B 15/30; G16B 40/00

(52) U.S. Cl. CPC G16B 15/30; G16B 40/00

(57) ABSTRACT

Reinforcement learning is coupled to a deep generative model based on a three-dimensional scaffold model to generate drug candidates targeting a particular protein, building up atom or functional groups from a starting core scaffold. A reward function can use parallel graph neural network models and take the particular protein into account when calculating reward based on criteria such as binding, synthetic accessibility, and the like. In an agent-critic reinforcement learning model, the agent learns to build molecules in three-dimensional space while optimizing the criteria.

### CROSS-REFERENCE TO RELATED APPLICATIONS

This application claims the benefit of U.S. Provisional Application No. 63/409,006, filed Sep. 22, 2022, and U.S. Provisional Application No. 63/461,531, filed Apr. 24, 2023, all of which are incorporated by reference herein.

### STATEMENT REGARDING FEDERALLY SPONSORED RESEARCH OR DEVELOPMENT

This invention was made with Government support under Contract DE-AC0576RL01830 awarded by the U.S. Department of Energy. The Government has certain rights in the invention.

### FIELD

The field generally relates to reinforcement learning in a drug discovery context.

### BACKGROUND

Current drug discovery techniques are intuition-derived, hampered by slow iterative design-test cycles due to computational challenges, and ultimately limited by the expertise of a chemist, leading to bottlenecks. Accordingly, there remains a need for improved drug discovery technologies.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, a computing system comprises one or more processors; memory; an internal three-dimensional representation of an input target protein; a generative machine learning model comprising an agent and a critic function; and a training environment configured to execute a plurality of training episodes, wherein for a given training episode, the training environment performs (a)-(c): (a) receiving a three-dimensional representation of a molecular scaffold as an internal three-dimensional representation of a molecule under construction; (b) with an agent in a generative machine learning model, iteratively adding a representation of one or more atoms to the internal three-dimensional representation of the molecule under construction; and (c) with a critic function in the generative machine learning model, calculating a reward, wherein calculating the reward comprises evaluating one or more binding criteria for the molecule under construction and the input target protein via the internal three-dimensional representation of the molecule under construction and the internal three-dimensional representation of the input target protein in a three-dimensional context; wherein the training environment is further configured to perform training the generative machine learning model with the reward.

In another embodiment, a computer-implemented method comprises receiving a three-dimensional representation of a molecular scaffold as an internal three-dimensional representation of a molecule under construction; with an agent in a generative machine learning model, iteratively adding a representation of one or more atoms to the internal three-dimensional representation of the molecule under construction, wherein the agent in the generative machine learning model is trained with a reinforcement learning process comprising rewards provided by a critic function evaluating one or more binding criteria of a plurality of training molecules under construction represented by internal three-dimensional representations of the training molecules under construction with an input target protein represented by an internal three-dimensional representation of the input target protein in a three-dimensional context; reaching a stopping point; and outputting the internal three-dimensional representation of the molecule under construction.

In another embodiment, one or more non-transitory computer-readable media comprise computer-executable instructions that, when executed by a computing system, cause the computing system to perform operations comprising: receiving a target-protein-agnostic generative machine learning model comprising an agent and a critic; receiving an internal three-dimensional representation of a target protein; training the target-protein-agnostic generative machine learning model with reinforcement learning, wherein the training comprises performing (a)-(c) for a plurality of training episodes: (a) receiving a three-dimensional representation of a molecular scaffold as an internal three-dimensional representation of a molecule under construction; (b) with an agent in a generative machine learning model, iteratively adding a representation of one or more atoms to the internal three-dimensional representation of the molecule under construction; and (c) with a critic function in the generative machine learning model, calculating a reward, wherein calculating the reward comprises evaluating one or more molecular properties comprising one or more binding criteria for the molecule under construction and the target protein via the internal three-dimensional representation of the molecule under construction and the internal three-dimensional representation of the target protein in a three-dimensional context; (d) communicating the reward to the agent, and modifying the agent according to the reward; whereby the target-protein-agnostic generative machine learning model is trained as a target-protein-specific generative machine learning model operable to generate three-dimensional representations of molecules for binding to the target protein.

As described herein, a variety of other features and advantages can be incorporated into the technologies as desired.

### BRIEF DESCRIPTION OF THE DRAWINGS

FIG. 1 is a block diagram of an example system implementing drug discovery via reinforcement learning with three-dimensional modeling.

FIG. 2 is a flowchart of an example method of implementing drug discovery via reinforcement learning with three-dimensional modeling.

FIG. 3 is a block diagram of an example system implementing a three-dimensional molecule representation framework.

FIG. 4 is a flowchart of an example method of implementing drug discovery via reinforcement learning with three-dimensional modeling from a training perspective.

FIG. 5 is a flowchart of an example method of implementing drug discovery via reinforcement learning with three-dimensional modeling from a prediction perspective.

FIG. 6 is a block diagram of an example system generating a reward for use during drug discovery via reinforcement learning with three-dimensional modeling.

FIG. 7 is a block diagram of an example process implementing drug discovery via reinforcement learning with three-dimensional modeling.

FIG. 8 is a block diagram of an example agent-environment-critic system implementing drug discovery via reinforcement learning with three-dimensional modeling.

FIG. 9 is a block diagram of an example alternative architecture implementing the technologies described herein.

FIG. 10 is a block diagram of an overview of decoding protein interaction with domain-aware machine learning that can be used to implement the technologies described herein.

FIG. 11 is a graph of example training and validation loss.

FIG. 12 is a graph comparing results for generated compounds.

FIG. 13A-D are graphs comparing properties of molecules generated with the technologies described herein.

FIGS. 14A and 14B are two-dimensional structure diagrams of top candidates generated with the technologies described herein with two different reward functions.

FIGS. 15A and 15B are three-dimensional structure diagrams of top candidates generated with the technologies described herein with two different reward functions.

FIG. 16 is a graph comparing properties of molecules generated with the technologies described herein.

FIG. 17A-D are graphs comparing properties of molecules produced from the technologies described herein against experimentally identified active compounds for Mₚᵣₒ target.

FIG. 18A shows a three-dimensional structure representation of M^{pro}. FIG. 18B shows a computational workflow for targeted design.

FIG. 19 are violin plots of AI-generated covalent compounds.

FIG. 20 are graphs showing biochemical characterization of the kinetics of M^{Pro} inhibition.

FIGS. 21A-E shows various properties of results.

FIGS. 22A-C shows room-temperature X-ray crystal structure of SARS-CoV-2 M^{pro}.

FIG. 23 shows conformational changes observed upon ligand binding.

FIG. 24 shows a computational covalent docking workflow.

FIG. 25 shows a 3D-scaffold deep learning (DL) framework.

FIG. 26 is a block diagram of an example computing system in which described embodiments can be implemented.

FIG. 27 is a block diagram of an example cloud computing environment that can be used in conjunction with the technologies described herein.

### DETAILED DESCRIPTION

### Example 1—Overview

In the context of drug discovery, a protein-ligand interaction refers to the binding or interaction between a target protein and a small molecule called a ligand. The target protein is usually a specific protein involved in a disease process or pathway, and the ligand is a potential drug candidate or compound that has the potential to bind to and modulate the function of the protein. By designing ligands that bind tightly and specifically to their target proteins, researchers aim to develop drugs that can modulate the activity of the target protein, leading to therapeutic effects and potential treatments for various diseases.

Efficient design and discovery of target-driven molecules is a critical step in facilitating lead optimization in drug discovery. Current approaches to develop molecules for a target protein are intuition-driven, hampered by slow iterative design-test cycles due to computational challenges in utilizing three-dimensional data, and ultimately limited by the expertise of a chemist, leading to bottlenecks in molecular design.

Instead, a novel framework couples reinforcement learning (RL) to a deep generative model based on a three-dimensional internal representation to generate target candidates specific to a protein building up atom-by-atom from a starting core scaffold.

The technologies described herein provide an efficient way to optimize key features by a reward function within a protein pocket using parallel graph neural network models.

The agent learns to build molecules in three-dimensional space while optimizing criteria such as activity, binding, affinity, potency, and synthetic accessibility of the candidates generated for infectious disease target proteins. The technologies can serve as an interpretable artificial intelligence (AI) tool for lead optimization with goals such as activity, potency, and biophysical properties.

As described herein, a generative learning technique can be applied in a drug discovery context by integrating a specific target protein into the generative learning process via reinforcement learning in an actor-critic model.

Other techniques such as compound (e.g., multi-objective) rewards can be used as described herein.

The described technologies thus offer considerable improvements over conventional drug discovery techniques.

### Example 2—Example System Implementing Drug Discovery Via Reinforcement Learning with Three-Dimensional Modeling

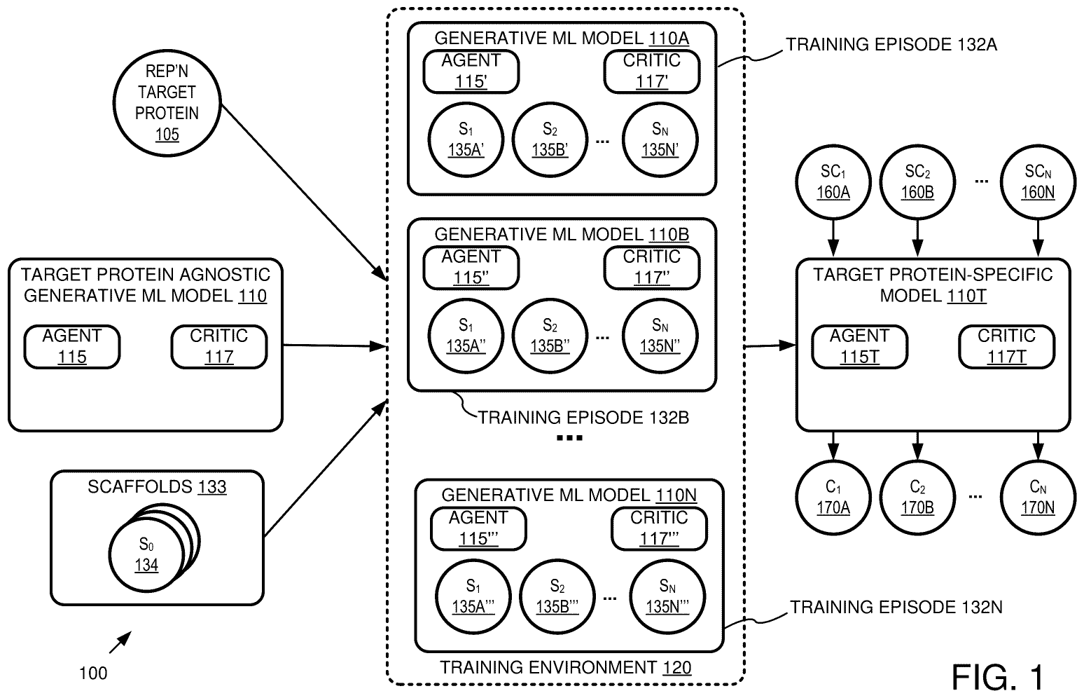

FIG. 1 is a block diagram of an example system 100 implementing drug discovery via reinforcement learning with three-dimensional modeling. In the example, the system 100 comprises a three-dimensional representation 105 of a target protein used as input to a training environment 120 for a generative machine learning model 110. As shown, the model 110 can start out as a target-protein-agnostic model and comprises an agent 115 and a critic function 117 that are trained as part of the training environment 120 to generate molecules that target the target protein represented by the three-dimensional representation 105.

The training environment 120 is configured to execute a plurality of training episodes 132A-N as shown. A given training episode 132A with the model 110A involves an agent 115′ that generates an internal three-dimensional representation of a molecule under construction starting with a scaffold 134 (S₀) and adding atom groups to the representation of the molecule under construction as it progresses from state S₁ 135A′ to Sₙ 135N′. A reward function provided by the critic function 117′ results in a modified agent 115′ (e.g., with a modified policy) that is able to generate future representations of molecules (e.g., 135N″, 135N″′, and so forth) that better meet the criteria incorporated into the reward function. The critic 117′ can also be modified to result in a critic (e.g., 117″, 117″′, and so forth) better able to assess the criteria over time. In practice, the training environment generates a target protein specific generative machine learning model 110T that is configured to receive representations of input scaffolds 160A-N and generate representations of candidate molecules 170A-N that satisfy the criteria as described herein.

After the first training episode 132A with the model 110A, which builds the molecule representations 135A′, 135B′, . . . 135N′ with the agent 115′ and critic 117′, other training episodes 110B through 110N can be executed. Training episode 132B uses the partially trained model 110B to build the molecule representations 135A″-N″ with the agent 115″ and critic 117″. Training episode 132N uses the model 110N to build the molecule representations 135A″′-N″′ with the agent 115″′ and critic 117″′. In practice, a training epoch can comprise training over multiple training episodes. Multiple epochs are possible.

Due to the inclusion of the representation of the target protein 105, the model 110T is trained by reinforcement learning comprising rewards provided by a critic function so that it generates representations of candidate molecules that meet the goal criteria incorporated into the critic 117. As described herein, such criteria can comprise binding criteria (e.g., binding probability, binding affinity, or the like), synthetic accessibility (SA), and the like. The critic function 117 can continue to use the three-dimensional representation 105 of the input target protein when calculating the reward function. Accordingly, the molecule representations 170A-N generated by the model 110T can be used as drug discovery candidates for the protein represented by the representation 105.

Thus, for a given training episode, the training environment 120 receives a three-dimensional representation 133 of a molecular scaffold as an internal three-dimensional representation of a molecule under construction; with the agent (e.g., 115′) in the generative machine learning model (e.g., 110A), iteratively adds a representation of one or more atoms to the internal three-dimensional representation of the molecule under construction (e.g., 135A′-N′); and with a critic function (e.g., 117′) in the generative machine learning model (e.g., 110A), calculates a reward, wherein calculating the reward comprises evaluating one or more binding criteria of the molecule under construction represented by internal three-dimensional representation.

The training environment 120 is configured to perform training the generative machine learning model with the reward generated by the critic (e.g., 117, 117′, 117″, or 117″′). Training with the reward can comprise calculating gradients and tuning weights of the generative machine learning model. The critic can be pretrained on known protein-ligand complexes and be static in the context of training the generative model. However, the critic can be optimized towards a target system by training the model for a specific protein.

Training can continue until stopping criteria are reached, such as whether the model 110 continues to learn (e.g., the predictive power increases) based on benchmarked performance. For example, training loss and validation loss of the model can be evaluated as described herein. Training can also monitor the reward function to determine an appropriate time to stop training. A threshold reward function value can be set for one or more of the criteria described herein, and training can stop responsive to determining that the critic function of the model is outputting rewards that meet the threshold.

Any of the systems herein, including the system 100, can comprise at least one hardware processor and at least one memory coupled to the at least one hardware processor.

The system 100 can also comprise one or more non-transitory computer-readable media having stored therein computer-executable instructions that, when executed by the computing system, cause the computing system to perform any of the methods described herein.

In practice, the systems shown herein, such as system 100, can vary in complexity, with additional functionality, more complex components, and the like. For example, the training environment 120 can execute significantly more training episodes 132A-N, and predictions by the trained model 110T can be validated. There can be additional functionality within the training process. Additional components can be included to implement security, redundancy, load balancing, report design, and the like.

Further, although the system 100 is shown as having a definitive, training/trained boundary, in practice, the system 100 can iterate over a plurality of training episodes until a goal optimization level has been reached (e.g., training episodes are repeated). At such a point, a model performing at a threshold level can be adopted as the trained model for making predictions. However, the predictions of a model during the training process can be used as candidates. Therefore, the boundary between training and trained need not be definitive.

Also, “n” is used several times in FIG. 1; in practice, the values for n may vary depending on context. For example, it is not necessary that the number of training episodes match the number of states, the number of input scaffolds, or the like.

The described computing systems can be networked via wired or wireless network connections, including the Internet. Alternatively, systems can be connected through an intranet connection (e.g., in a corporate environment, government environment, or the like).

The system 100 and any of the other systems described herein can be implemented in conjunction with any of the hardware components described herein, such as the computing systems described below (e.g., processing units, memory, and the like). In any of the examples herein, the representation 105, model 110, scaffolds 133, training environment 120, candidates 170A-B, and the like can be stored in one or more computer-readable storage media or computer-readable storage devices. The technologies described herein can be generic to the specifics of operating systems or hardware and can be applied in any variety of environments to take advantage of the described features.

### Example 3—Example Method Implementing Drug Discovery Via Reinforcement Learning with Three-Dimensional Modeling

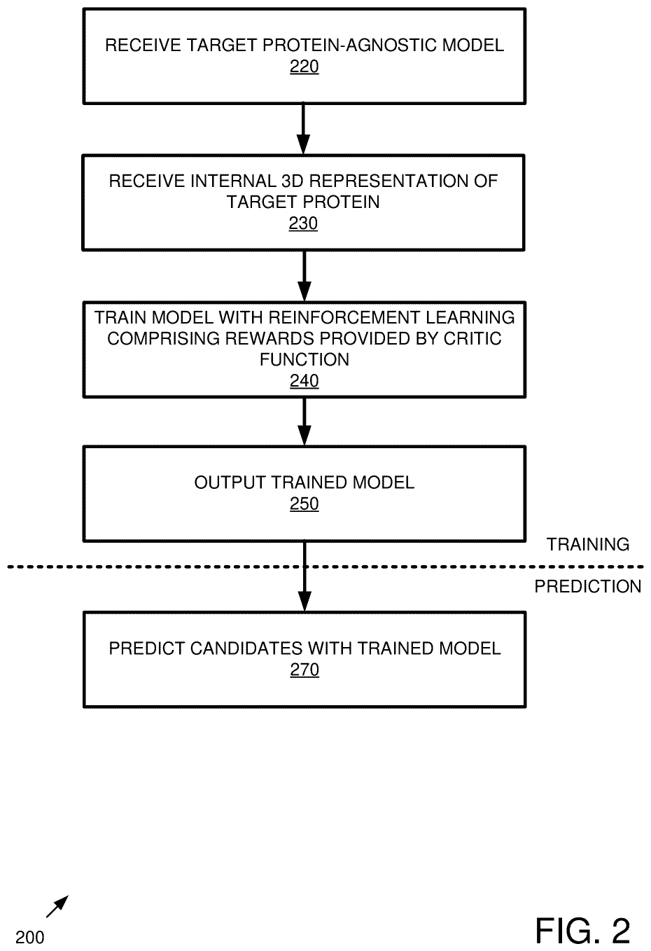

FIG. 2 is a flowchart of an example method 200 of implementing drug discovery via reinforcement learning with three-dimensional modeling and can be performed, for example, by the system of FIG. 1. The automated nature of the method 200 can be used in a variety of situations such as assisting in drug discovery, generating drug candidates, target-protein-specific hit identification, lead optimization, or the like.

In the example, at 220, a target-protein-agnostic generative machine learning model is received. As described herein, such a model comprises an actor and a critic that cooperate in a reinforcement learning scenario.

At 230, an internal three-dimensional representation of a target protein is received.

At 240, the model is trained with reinforcement learning comprising rewards provided by a critic function of the model. As described herein, the critic can provide a reward based on criteria such as binding criteria, synthetic accessibility, and the like. As described herein, training can take the form of iterating through training episodes that generate respective molecules output from the generative machine learning model, which is transformed into a protein-specific generative machine learning model by the training process.

At 250, the trained model is output.

At 270, drug candidates are predicted with the trained model.

As described herein, the process of training can be iterative, and a clear demarcation between training and prediction need not be implemented. Training can continue until a level of desired model performance is met. Then, the generated candidates can be used as output for further research or in vivo/vitro testing.

The method 200 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies. For example, receiving a reward can be described as sending a reward depending on perspective.

### Example 4—Example Protein Applicability

In any of the examples herein, the technologies can be used in a drug discovery context to target any of a number of protein targets. Thus, the term “protein-agnostic” or “target-protein-agnostic” is sometimes used to describe scenarios where a plurality of different proteins can be targeted. As described herein, the generative model can be initially protein agnostic and then be trained to generate molecules for a given protein via training episodes that implement a critic with reference to the given protein target. In such a case, binding to the protein in a three-dimensional context can be evaluated during training, training the generative model to learn how to generate molecules that bind to the given protein via reinforcement learning.

As a result, the agent and critic of the model described herein can be configured to operate for a plurality of input target proteins.

### Example 5—Example Training Environment

In any of the examples herein, a training environment can comprise a computing system configured to execute software tools that perform the training process. As described herein, a target-protein-agnostic generative machine learning model can be trained in the training environment by executing a plurality of training episodes that generate candidate molecules with an agent. A critic provides a reward function to the agent based on criteria as described herein.

The training environment can include tools for selecting parameters for the training episodes, such as an input molecular scaffold and receiving a three-dimensional representation of the molecular scaffold. The training environment can also orchestrate the training episodes by executing the generative machine learning model with the input scaffold and observing the results. A stopping condition can be monitored to determine when to stop training as described herein.

### Example 6—Example Criteria

In any of the examples herein, the criteria applied during learning by a critic function can include a variety of goal criteria. In practice, one or more binding criteria (e.g., between the target protein and the molecule under construction) such as binding probability, binding affinity, and the like can be evaluated and used. One or more molecular properties comprising one or more binding criteria can be evaluated and used. Training can thus be based on evaluating the current candidate and protein in the context previous protein-ligand interaction data. Other criteria such as synthetic accessibility of the molecule under construction can also be applied.

Although a single criterion can be used to calculate a reward, as described herein, a compound reward can comprise more than one criterion. For example, the binding criteria and synthetic accessibility of the molecule under construction can be combined.

As described herein, criteria can be predicted by predicting biophysical properties (e.g., how a molecule binds to a protein). By providing a reward function that incorporates the desired criteria to the agent, the agent learns to build molecules that fulfil the stated criteria. If there is a negative reward for a molecule, the agent will attempt to avoid such molecules in the future.

### Example 7—Example Optimization of Criteria

In some examples, the terms “optimize,” “optimization,” or the like are used. However, such language does not require that the best solution be found. As used herein, “optimized” does not necessarily refer to a “best” approach and includes a variety of approaches that find an improved solution (e.g., compared to previous solutions).

### Example 8—Example Drug Discovery Candidates

In any of the examples herein, the output of the target protein specific generative machine learning model can serve as drug discovery candidates that target the target protein of a given disease or pathogen. In practice, further research or clinical work may be performed to refine a candidate, but as shown herein, the model does generate candidates that exhibit verifiable characteristics of drug-like molecules.

### Example 9—Example Three-Dimensional Molecule Representation Framework

In any of the examples herein, a three-dimensional molecule representation framework configured to generate and represent three-dimensional representations of molecules under construction can be used and incorporated into the agent described herein. One such framework is described in “3D-Scaffold: A Deep Learning Framework to Generate 3D Coordinates of Drug-like Molecules with Desired Scaffolds,” Joshi et al., J. Phys. Chem. B 2021, 125, 12166-12176 (2021), which is hereby incorporated by reference herein along with any supplemental materials. Such a framework can store a three-dimensional representation of a molecule as an internal (e.g., in silico) three-dimensional representation of a molecule under construction.

The framework can accept an input scaffold represented as three-dimensional coordinates indicating the types of atoms and three-dimensional structure of the scaffold and iteratively generate one or more atoms to the scaffold as a molecule under construction.

Such a framework can comprise a domain-aware generative framework that generates valid, unique, novel, and synthesizable molecules that have drug-like properties similar to molecules in a training set. As described herein, generation can be trained with a target protein so that drug candidates are generated by the framework.

In practice, the molecule under construction can be represented internally as a collection of atoms having atom types and placed in three-dimensional space at respective locations. Three-dimensional modeling representation of a molecule under construction can thus be implemented.

In practice, the molecule is called a “molecule under construction,” but the molecule can be considered “constructed” after a stopping point (e.g., upon reaching stopping criteria, such as binding criteria or the like). The constructed molecule can then be used as a candidate for drug discovery as described herein.

Also, sometimes the shorthand “molecule” is used when the internal representation of the molecule is intended. As described herein, the training process operates by calculating criteria such as protein binding based on software models that incorporate a three-dimensional representation of the protein and the molecule under construction.

### Example 10—Example Agent

In any of the examples herein, an agent incorporated into the generative machine learning model can implement the three-dimensional molecule representation framework described herein. As noted, such a framework can comprise a neural network implementing feature learning and atom placement. Thus, the agent can be configured to perform atom placement.

Feature learning in this context focuses on the molecular features, which include properties like functional groups, molecular weight, bond type (e.g., single, double, triple), atom position, bond angle, atom type (element), functional groups, and the like. The same features can be used for proteins and scaffolds.

Embedding and interaction layers can be used to extract and update rotationally and translationally invariant atom-wide features that capture the chemical environment of the molecule under construction. Such features can be used to predict distributions for a type of next atom to be added and its three-dimensional coordinates. Such distributions can then be used to randomly select the atom type and its placement, which is then added to the molecule under construction. As described herein, functional groups can also be supported as the molecule is built up atom-by-atom or atom group-by-atom group.

In practice, the agent is configured to choose a type of the one or more atoms based on the current state based on reward provided by the critic. It does so by adjusting its policy over time to increase the probabilities of actions that lead to higher rewards and decrease the probabilities of actions that result in lower rewards.

As an example, described herein, the agent can be configured to choose of the one or more atoms based on the following probability, which is described in more detail herein:

$${{P}_{Θ} ( {s}_{t} )}^{type} = - log ( {\hat{p}}_{type}^{{Z}_{next}} ) .$$

The agent can be configured to add a representation of the one or more atoms to the internal three-dimensional representation of the molecule under construction at a distance from an already-placed atom represented in the internal three-dimensional representation of the molecule, wherein the distance is based on a probability assigned by the three-dimensional molecule representation framework. A distribution of such probabilities can then be used to randomly select the atom placement. Like atom type, atom placement can be based on a policy that evolves over time in light of reward provided by the critic.

As an example herein, the agent can be configured to choose the distance based on the following probability, which is described in more detail herein:

$${{P}_{Θ} ( {s}_{t} )}^{dist} = {\sum }_{j = 1}^{N} {\sum }_{b ∈ B} {q}_{j}^{b} log ( {\hat{p}}_{j}^{b} ) .$$

### Example 11—Example Critic Function

In any of the examples herein, a critic function or (“critic”) incorporated into the generative machine learning model can implement a reward function that serves to train the model as part of reinforcement learning. As described herein, various criteria can be implemented in the critic function, and a compound reward can be supported where the reward incorporates more than one criterion.

As described herein, the critic can comprise parallel graph neural networks configured to determine binding probability indicating whether the molecule under construction binds with the input target protein. Such probability is calculated based on the internal three-dimensional representations of the molecule under construction and the input target protein.

The critic can be configured to assess the molecule under construction a plurality of times for a given training episode. For example, the critic can assess the molecule under construction each time one or more atoms is added (e.g., each time the agent acts).

The critic can be configured to score multiple times per episode or only at the end. The critic may sometimes be unable to properly score a partial molecule; user configuration can indicate whether to pass a completed molecule to the critic or to allow in-progress molecules to be scored. The final molecule can then be scored by the critic.

### Example 12—Example Three-Dimensional Molecule Representation Framework Implementation

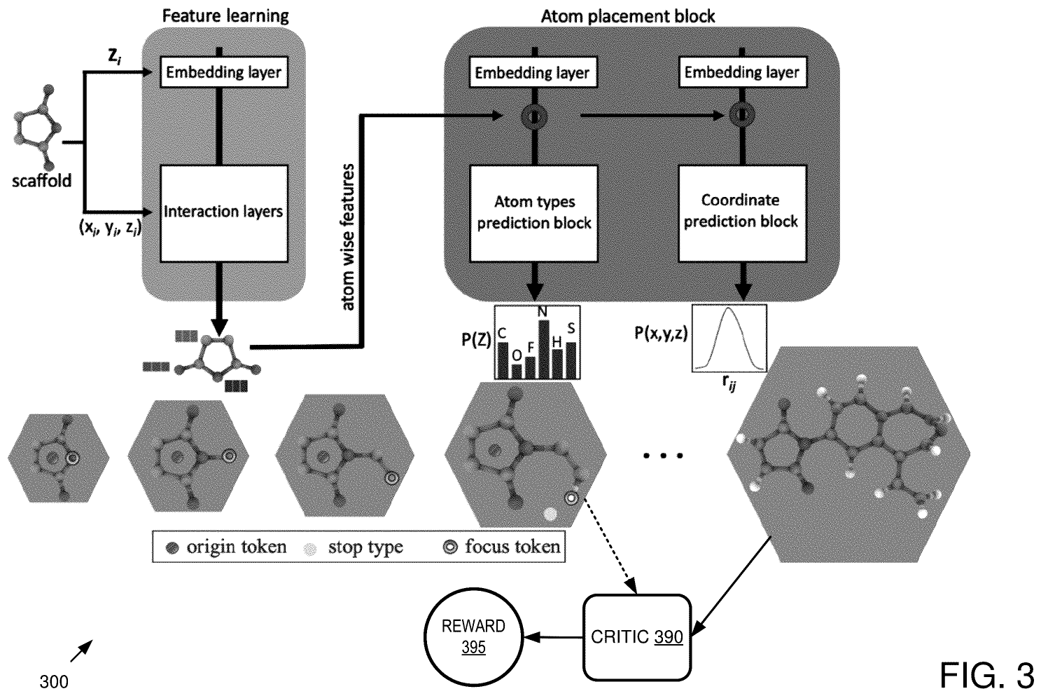

FIG. 3 is a block diagram of an example system 300 implementing a three-dimensional molecule representation framework that can be used for de novo therapeutic candidate design. The framework can be built on a deep neural network that builds molecules around a desired scaffold. In the example, molecule design is broken into two major blocks—feature learning and atom placement. In the feature learning block, embedding and interaction layers (e.g., of SchNet) can be used to extract and update rotationally and translationally invariant atom-wise features that capture the chemical environment of an unfinished molecule.

A neural network can utilize continuous-filter convolution layers as a way to learn robust representations of molecules starting from the positions of atoms and corresponding nuclear charges.

In the atom placement block, the extracted features can be used to predict distributions of the type of next atom and its three-dimensional coordinates, where the latter distribution is constructed from predictions of pairwise distances between the next atom and the preceding atoms.

To do actual placement of the next atom in three-dimensional space, a distribution on a small grid with candidate positions focused on one of the preceding atoms is constructed from the predicted pairwise distances.

The process can start with an input scaffold. The above procedure can be repeated iteratively to build a complete molecule with the desired scaffold. Due to the scaffold starting point, the framework is sometimes called a “3D Scaffold framework.” As shown, the framework can be used to generate molecules under construction that are evaluated by the critic function 390 to generate a reward 395 that can be used as feedback to control generation of future atoms, molecules, and the like.

The molecule panels of FIG. 3 shows the scaffold-based molecular generation scheme, where the origin token, focus token, and stop type aid the generation of the molecules from scaffolds. The framework can be configured to generate only atoms (e.g., of particular atom types) in three-dimensional space; for visual clarity, the atoms can be shown as connected with bonds as shown.

The generation process can be aided by two auxiliary tokens with unique, artificial types: the origin and focus tokens.

### Example 13—Example Atom Types

In any of the examples herein, the molecule under construction is represented internally with a three-dimensional representation that includes atom types. In practice, although an “atom-by-atom” terminology is used, an atom group can be represented and added during molecule generation.

In practice, various atom types can be carbon (C), oxygen (O), fluorine (F), nitrogen (N), hydrogen (H), sulfur (S), and the like. However, functional groups can be treated as an atom type and added during molecule generation; such functional groups can be represented by special symbols or their constituent atoms. Thus, generation can add one or more atoms (e.g., an atom group) in an iterative function as part of the generative process.

### Example 14—Example Three-Dimensional Representation of a Target Protein

In any of the examples herein, the technologies can be employed for drug discovery in the context of a target protein. Molecular design for a specific protein target can be a critical aspect of drug development and can be highly effective in treating diseases, pathogens, or altering disease pathways. After the structure of the target protein is understood, the workflow can begin designing a drug that can interact with it.

One such example is the Sars-CoV-2 Main protease (M^{pro}) protein. It is a known target for therapeutic design because its inhibition effective prevents the protein from infecting the host organism. So, protein-directed inhibition can be important because eliminating the protein function prevents the protein from completing its role (e.g., viral function, biological function, or the like).

In practice, the target protein is represented internally in a computing system as a three-dimensional representation indicating the types of atoms and positions of the atoms in the protein. Three-dimensional modeling of a target protein can thus be implemented in a graph. The protein target's three-dimensional structure can be represented in Graph Neural Networks (GNNs), where nodes represent amino acids (e.g., residues) and edges represent connections between them. The adjacency matrix often represents the distances or interactions between different amino acids in the protein. Edges can also be weighted based on the strength of the interaction or the distance between the connected nodes. GNNs can then process the graph to learn complex patters in the protein's structure, which can be used for various prediction tasks.

A two-dimensional representation can be part of the three-dimensional representation.

### Example 15—Example Machine Learning Model

In any of the examples herein, a machine learning model can be used to generate molecules under construction, which serve as representations of drug candidates as described herein. Such models are stored in computer-readable media and are executable with input data to generate an automated prediction, namely atom type and position as described herein.

Other elements of the technology can be implemented as machine learning models. For example, the three-dimensional molecule representation framework can be implemented as a deep neural network trained end-to-end with backpropagation using ground truth types and pairwise distances of atoms in training molecules.

The critic function can be implemented as a machine learning model trained to predict criteria such as binding criteria, synthetic accessibility, and the like.

As described herein, inputs to the model can be the 3D coordinates of the molecules in the training set and the SMILES strings of the corresponding scaffolds. Other arrangements are possible.

### Example 16—Example Training Perspective

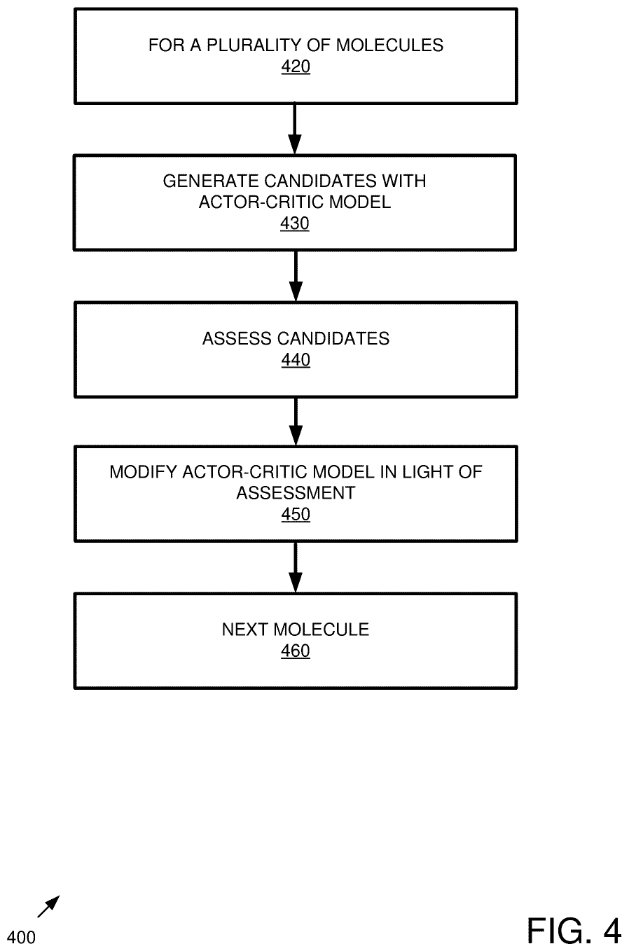

FIG. 4 is a flowchart of an example method 400 of implementing drug discovery via reinforcement learning with three-dimensional modeling from a training perspective. The method 400 can be used to implement training 240 of FIG. 2.

In the example, the process is iterated 420 for a plurality of molecules under construction in respective training episodes.

At 430, candidates are generated with an actor-critic model as described herein. The critic can participate in the placement of atom groups.

At 440, a candidate is assessed. In practice, a plurality of candidates can be assessed in a batch.

At 450, the actor-critic model is modified in light of the assessment. For example, gradients can be calculated and model weights can be tuned.

At 460, the method can move on to the next molecule.

Training can continue until a level of desired performance is reached (e.g., a threshold level of criteria fulfilment is observed).

### Example 17—Example Prediction Perspective

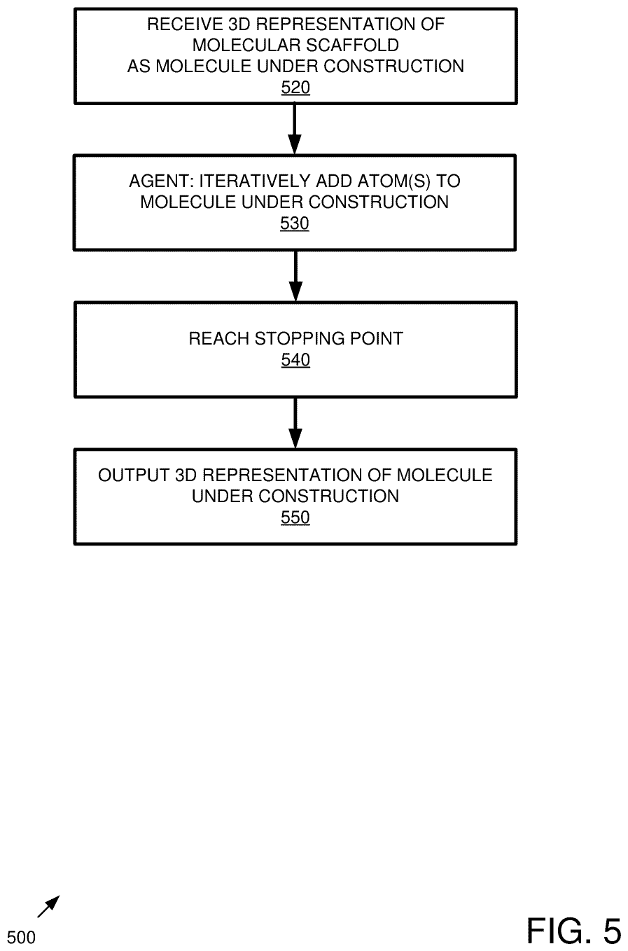

FIG. 5 is a flowchart of an example method of implementing drug discovery via reinforcement learning with three-dimensional modeling from a prediction perspective and can be used, for example, to implement prediction 270 of FIG. 2. In the context of generative drug discovery, the terms “predict” and “generate” can be used interchangeably. The model is predicting a molecule that will fulfill the criteria implemented in the critic function, and the generated prediction can be used as a generated candidate molecule for drug discovery purposes.

Given a generative machine learning model that has been trained using a given target protein, the method 500 can be used to generate candidate molecules for the target protein.

At 520, a three-dimensional representation of a molecular scaffold is received as a molecule under construction. As described herein, the scaffold can serve as a starting point for the molecule under construction.

At 530, an agent of a generative machine learning model iteratively adds one or more atoms to the internal three-dimensional representation of the molecule under construction. As described herein, an atom type and atom location are selected and added to the molecule under construction. The agent in the generative machine can be trained with a reinforcement learning process comprising rewards provided by a critic function evaluating one or more binding criteria of a plurality of training molecules under construction represented by internal three-dimensional representations of the training molecules under construction with an input target protein represented by an internal three-dimensional representation (e.g., of the input target protein in a three-dimensional context).

As described herein, given the sequential nature of building the molecule, the critic function can assess the molecule as construction takes place to guide the actor in the generation process. The reward can be communicated to the agent, and the agent is modified according to the reward.

In practice, the agent operates based on the current policy. The reward provides feedback to the agent indicating desirability or utility of a particular action in a state. The agent's objective is to maximize the expected cumulative reward over time. The agent can update its policy to improve its future action selection based on the reward. Policy gradients or temporal difference learning can be used.

Policy gradient optimization can be used. As the reward is calculated, the gradient is calculated and back propagation is applied to alter the weights and biases of the Agent. Application of reinforcement learning can be implemented. Tasks can include calculating the reward, using the reward to calculate the gradient, back propagating the gradient, updating the weights and biases, and the moving on to the next training episode. The update function can be an application of the REINFORCE policy gradient method, from reinforcement learning algorithms can derive. A goal is to optimize the policy, which is improved with increasing rewards.

The agent can be configured to choose an atom type based on a probability distribution as described herein. Similarly, the agent can be configured to choose an atom position based on a probability distribution. An atom of the atom type is then placed within the molecule at the atom position.

At 540, the generative process reaches a stopping point. For example, binding criteria or the like can indicate that the molecule has reached a desired level of performance, the size of the molecule can exceed a threshold, or the like.

At 550, a three-dimensional representation of the molecule under construction is output.

The resulting representation can be used to generate a physical molecule having a structure represented by the internal three-dimensional representation of the molecule under construction. A subject (e.g., human subject) can then be treated with such a molecule as part of drug therapy to modulate the target protein, resulting in disease treatment.

It is noted that the method 500 can mirror that used for training. In other words, the same acts can be performed for training or prediction, with the model expected to improve in performance during training.

### Example 18—Example Reward Generation

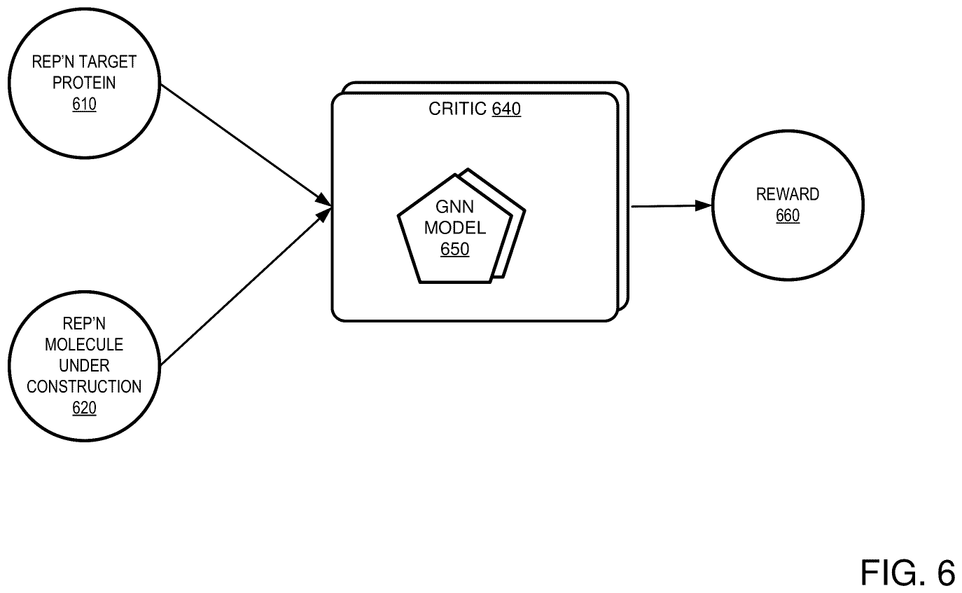

FIG. 6 is a block diagram of an example system generating a reward for use during drug discovery via reinforcement learning with three-dimensional modeling. In the example, a three-dimensional representation of the target protein 610 and a three-dimensional representation of a molecule under construction 620 can be received by a critic function 640 that generates a reward 660 that can be used for reinforcement learning purposes as described herein.

As shown, the critic model can use parallel graph neural networks 650 to predict criteria such as binding probability and binding affinity. Different criteria can be used, and a compound reward can be implemented where more than one criterion is implemented by the critic 640.

### Example 19—Example Prediction

In any of the examples herein, after the model is trained, it can be used to predict a candidate molecule (e.g., ligand) that modulates the activity of target protein in vivo (e.g., is assessed to modulate the target protein in vivo using protein modeling). As described herein, the technologies generate representations of molecules that have been assessed to be effective at binding with the protein.

As described herein, the technologies can thus generate the structure of a small molecule that fits in the pocket of the target protein.

### Example 20—Example System for Reinforcement Learning

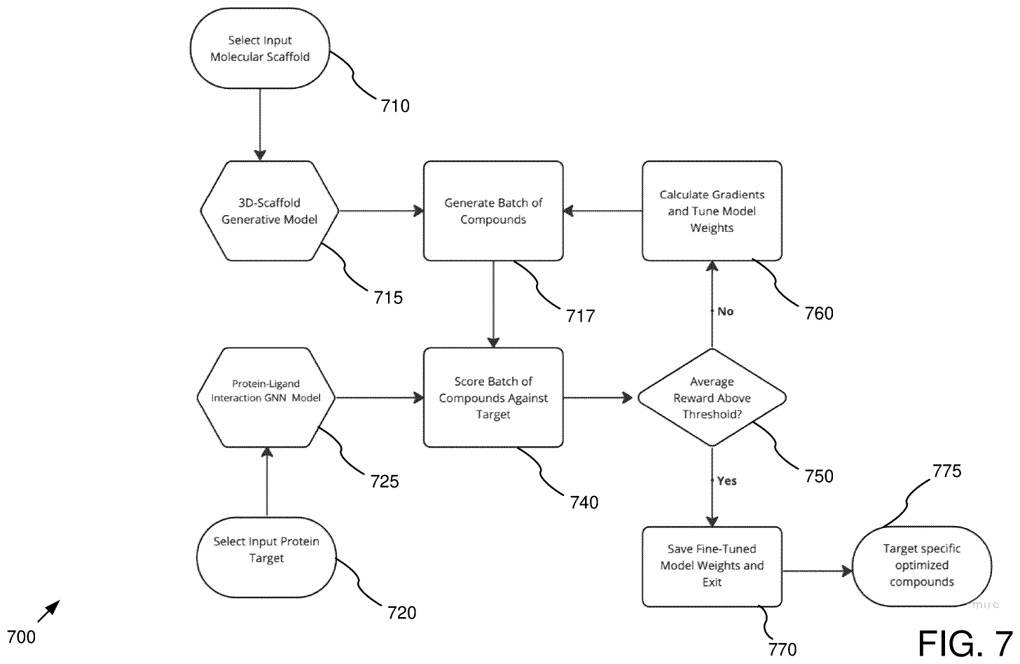

FIG. 7 is a block diagram of an example process implementing drug discovery via reinforcement learning with three-dimensional modeling that can be used in any of the examples herein. In the example, an input molecular scaffold 710 is selected as a starting state (S₀) for a molecule under construction and input to the three-dimensional-scaffold generative model 715, which can generate a batch of compounds 717 (e.g., molecules) as described herein.

Also an input target protein can be selected at 720, and a protein-ligand interaction graph neural network (GNN) model 725 can be used to generate scores 740 for the batch of compounds against the target protein.

At 750, if the average reward is not above a threshold, gradients can be calculated and model weights tuned at 760, and another batch of compounds with the tuned model can be generated at 717.

If the average reward is above the threshold, the fine-tuned model weights can be saved at 770. Then, target-protein-specific optimized compounds can be targeted at 775. For example, the last batch of compounds can be used, or additional compounds can be generated with the tuned or pretrained model.

### Example 21—Example Implementation: Problems

Recent advancements in machine learning and artificial intelligence have demonstrated the potential to revolutionize the drug design process by reducing the initial chemical search space in the early stages of discovery. Currently the potential chemical space is composed of more than 10⁶⁰ molecules; candidates with suitable activity against specific protein targets only narrows the search space significantly based on the critical fragments. The COVID-19 pandemic has brought a surge of interest to explore data-driven methods to better produce efficacious drug candidates. One of the most important factors in identifying new drug candidates is that the molecules possess optimized properties that allow them to effectively bind with a required target while also having minimal off-target effects.

If molecular generation is considered to be a controlled and dynamic step-by-step process, it is possible to reduce end products that possess these optimized properties. Such an approach allows one to formulate de novo drug design as a reinforcement learning problem and utilize algorithms that best learn a molecule's representation space based on the core moiety and its spatial interaction with the protein target. As an alternative reinforcement learning can provide a platform to create a highly efficient inverse molecular design artificial intelligence system capable of producing novel high performance molecules with domain targeted properties.

The novel technologies described herein can address the problem of generating molecules optimized for specific three-dimensional protein targets starting from core functional groups called “scaffolds.” Reinforcement-learning-based can be used to generate molecules in different ways. For example, a method utilizes a fragment-replacement-based approach to optimize existing SMILES strings for specific drug like properties while another method generates entirely new SMILES. Several current machine and reinforcement learning methods rely on the use of variational auto encoders, which learn the latent space representation of two-dimensional molecules to find the best representation. However, none of these methods take into consideration the three-dimensional protein structure during the generation phase. Some of these methods do test the drug-likeness post-generation against some given target protein, but the target is not directly involved in the reinforcement learning loop. When designing drug candidates that target specific proteins, learning how the molecule interacts with the proteins structure is invaluable to the generative process and can greatly assist in accelerating the decision making and automation in medicinal chemistry.

The technologies described herein can incorporate the protein structure into the reinforcement learning loop and also consider the three-dimensional structures of the generated compounds by constructing them atom-by-atom in three-dimensional space. To overcome the limitations of sequence-based representation of the target and the drug, a three-dimensional scaffold based molecular generation actor over a 2D SMILES generation agent can be used. The technologies described herein can use a three-dimensional scaffold-based generative model to make atom wise placements on the molecule starting from a desired scaffold.

As described herein, the technologies can use an agent-critic reinforcement learning method, where an agent learns to make decisions by performing certain actions in an environment to maximize some notion of cumulative reward. A scaffold-based generative model used to produce valid three-dimensional compounds can serve as the agent model. For the critic model, parallel graph neural networks (GNN_{P}) can be used as a binding probability predictor to evaluate whether the generated compound actively binds with a target protein. The technologies can take the three-dimensional structure of the protein pocket and the ligand and predict the probability of their interaction without any prior knowledge of the intermolecular interactions. The parallel graph neural networks model can use both the residue and three-dimensional atomic level representation of a protein as well as the three-dimensional atomic and bond level representation of the molecule.

### Example 22—Example Implementation: Experiments and Dataset

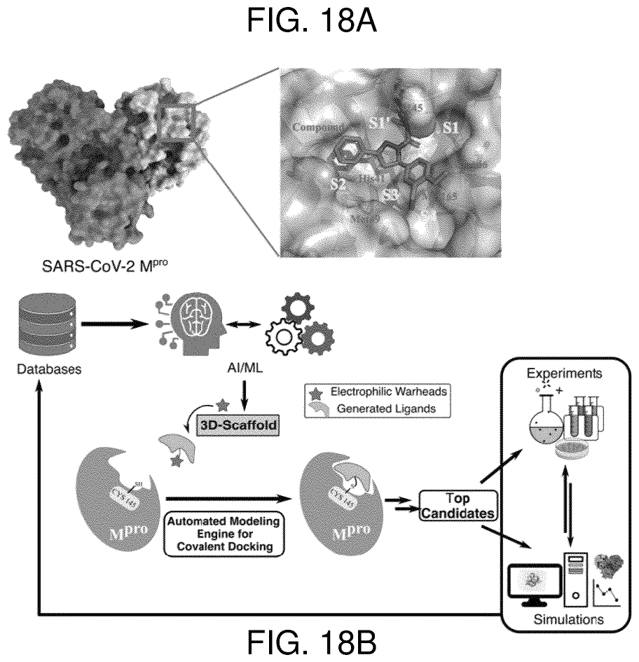

To demonstrate that the technologies are able to produce molecules for a given target protein, an implementation centering around creating drug-like molecules that interact with the SARS-CoV-2 Main Protease, Mₚᵣₒ was undertaken (see FIG. 18 and accompanying description herein). To train the model, a dataset of non-covalent inhibitors from the BindingDB data set and FDA approved drugs was used. The compounds were filtered based on biophysical and cheminformatics properties like IC₅₀, molecular weight, atom type, and functionality and have almost 10,000 unique scaffolds. To represent the definition of scaffold, Murcko scaffolds were used. High-throughput experimentation and the computer aided drug design results suggest that compounds containing the functional group piperazine are potent and have a higher affinity towards the Mₚᵣₒ target. However, there were no piperazine containing molecules in the initial data set used for training the three-dimensional scaffold model, but the technologies were still able to generate some molecules containing piperazine using the trained model. A smaller subset of the BindingDB data set was curated that possessed affinity towards protease-like targets and combined with the piperazine data set. Finally, the compounds were filtered based on their ability to bind with Mₚᵣₒ by doing an initial pass through the GNN_{P} model. With the goal of achieving better binding affinity potency and easily synthesizable compounds for the Mₚᵣₒ target, the initial screening helped choose proper compounds for training the reinforcement learning models so that they learned to produce similar or better compounds.

### Example 23—Example Implementation: Methods

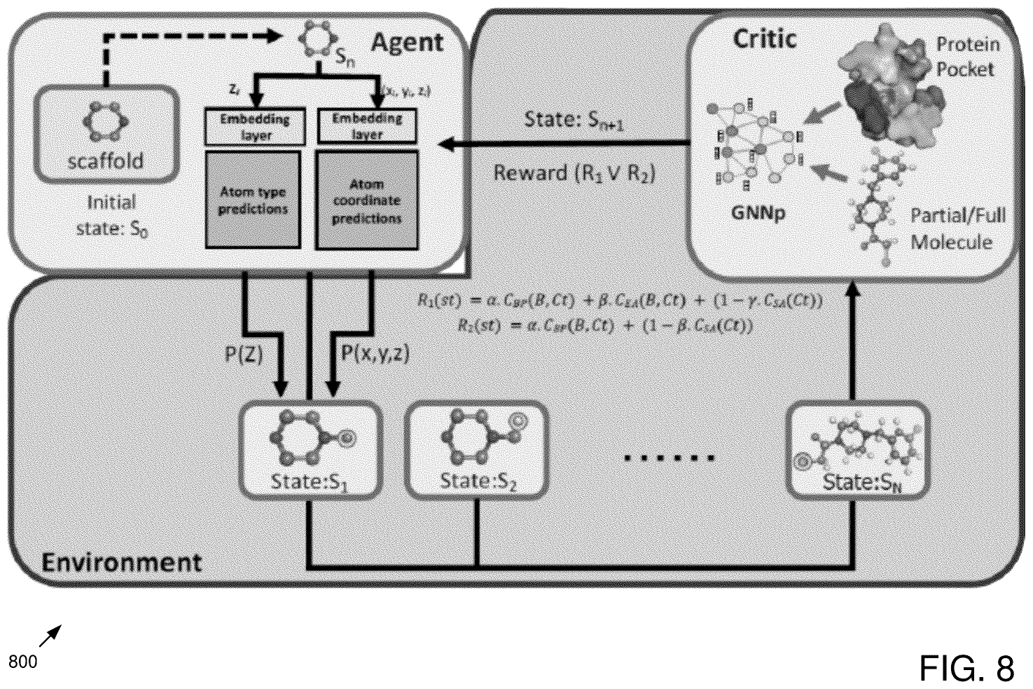

FIG. 8 is a block diagram of an example agent-environment-critic system 800 implementing drug discovery via reinforcement learning with three-dimensional modeling that can be used in any of the examples herein. The model is sometimes called “3D-MolGNN_{RL}” because it implements a three-dimensional molecular model via graph neural networks in a reinforcement learning context. In the example, a workflow is shown that highlights the interaction of agent and critic in the reinforcement learning model. The agent starts building the molecule from a starting scaffold (state S₀) and subsequently builds the molecule by choosing an atom based on the reward assigned by the critic for the intermediate state. The agent block serves as the actor, generating molecules to pass into the critic block to assess the performance.

As shown in FIG. 8, the reinforcement learning workflow can begin from training a three-dimensional scaffold molecule generator followed by optimizing the partially built candidates to the target of interest during the training steps. The neural network used in the three-dimensional scaffold framework for de novo drug candidate design can be broken into two major blocks feature learning and atom placement. In the feature learning block, the embedding and interaction layers of SchNet can be used to extract and update rotationally and translationally invariant atom-wise features that capture the chemical environment of an unfinished molecule. The extracted features can be used to predict distributions for the type of next atom and its three-dimensional coordinates, where the latter distribution is constructed from predictions of pairwise distances between the next atom and preceding atoms.

The process can be repeated successively to build a complete molecule with a desired scaffold. The partial molecule associated with a given step of the atom placement can be assessed by the critic. As part of the experiment, two critics were used simultaneously: the binding probability and binding affinity of the partial molecule and the target of interest along with the synthetic accessibility of the partial molecule. The binding probability was the outcome of a SoftMax layer which predicts the activity/inactivity of a protein-ligand complex. The binding affinity is measured in terms of Kᵢ and K_{d}, which refer to the inhibition and dissociation constants respectively and is given as -log(Kᵢ/K_{d}). The two critics can essentially use the same feature representation, but differ in terms of the data they have been trained on and also the label associated with the data. A detailed breakdown of the layers and hyperparameters associated with the agent and critic models is described hereinbelow.

The technologies can implement a reward function and parameters. The process of building the molecule can start from a desired scaffold associated with the selected core functionality. For a given action t which is a random selection and placement of the atom to the partial molecule, the three-dimensional scaffold model predicts the next possible atom that could be placed close to the center of mass to the partial molecule at step t−1 and transitions to state sₜ. At any step, let Zₙₑₓₜ be the ground truth type of the next atom and \hat{p}_{type}^{Z}^{ₙₑₓₜ} the probability that the model assigns to that type at the current step. This probability is converted to a negative log likelihood as P_{Θ(s}_{ₜ}₎^{type} (equation 3a). Here \hat{p}ⱼ^{b} is the probability that the model assigns to the distance between position of already placed atom and ground truth of the next atom to fall into distance bin b∈B at the current step. A distance bin can take the form of a predefined range of distances (e.g., respective different bins can represent distances of 0-1 angstroms, 1-2 angstroms, 2-3 angstroms, and the like). The distance-based probability is calculated using Gaussian expanded ground truth distances qⱼ^{b} and probability distribution of add-on placement \hat{p}ⱼ^{b} has shown in equation 3B. The agent can be trained to learn the latent space representation of the atom type and the atom's possible placement in three-dimensional space closest to the center of mass of the molecule generated so far. The overall probability for any action t is the summation of type-probability and distance-probability (Equation 2). The agent can be trained such that it learns to generate new compounds while optimizing the policy Π(Θ) (Equation 1). The policy can be defined as the difference between the action probabilities and the reward assigned by the critic at that step. In the example, the probability is the negative log-likelihood.

$$\prod ( Θ ) = {\sum }_{{s}_{t} ∈ S} ( {P}_{Θ} ( {s}_{t} ) - R ( {s}_{t} ) ) \qquad ( 1 )$$

$${P}_{Θ} ( {s}_{t} ) = {{P}_{Θ} ( {s}_{t} )}^{type} + {{P}_{Θ} ( {s}_{t} )}^{dist} \qquad ( 2 )$$

$${{P}_{Θ} ( {s}_{t} )}^{type} = - log ( {\hat{p}}_{type}^{{Z}_{next}} ) \qquad ( 3 a )$$

$${{P}_{Θ} ( {s}_{t} )}^{dist} = {\sum }_{j = 1}^{N} {\sum }_{b ∈ B} {q}_{j}^{b} log ( {\hat{p}}_{j}^{b} ) \qquad ( 3 b )$$

$${R}_{1} ( {s}_{t} ) = α . {C}_{BP} ( B , {C}_{t} ) + β . {C}_{EA} ( B , {C}_{t} ) + ( 1 - γ . {C}_{SA} ( {C}_{t} ) ) \qquad ( 4 )$$

$${R}_{2} ( {s}_{t} ) = α . {C}_{BP} ( B , {C}_{t} ) + ( 1 - β . {C}_{SA} ( {C}_{t} ) ) \qquad ( 5 )$$

In the example, two different reward functions were used: R₁(sₜ) and R₂(sₜ) associated with two independent experiments for the purposes of ablation hyperparameter testing. By removing portions of the reward function, one can see which attributes provide the best reward. The first reward is a function of binding probability, binding affinity of the target and the partially built molecule, and the synthetic accessibility of the molecule. While the second reward function uses only the binding probability and the synthetic accessibility score. Different weights were assigned to each of the components while calculating the rewards. For R₁ (Equation 5) weights of 0.5, 0.25, and 0.25 were used for binding probability, binding affinity, and synthetic accessibility, respectively. While for R₂ (Equation 4), weights of 0.75 and 0.25 were used for binding probability and synthetic accessibility respectively. The agent was trained for 150 epochs and the best trained model was chosen to generate compounds. The synthetic accessibility and binding affinity scores were scaled to be in between zero and one to match the scale of the binding probability. Additional details of the ablation study including some reward functions not shown in the example are described hereinbelow.

### Example 24—Example Implementation: Architecture

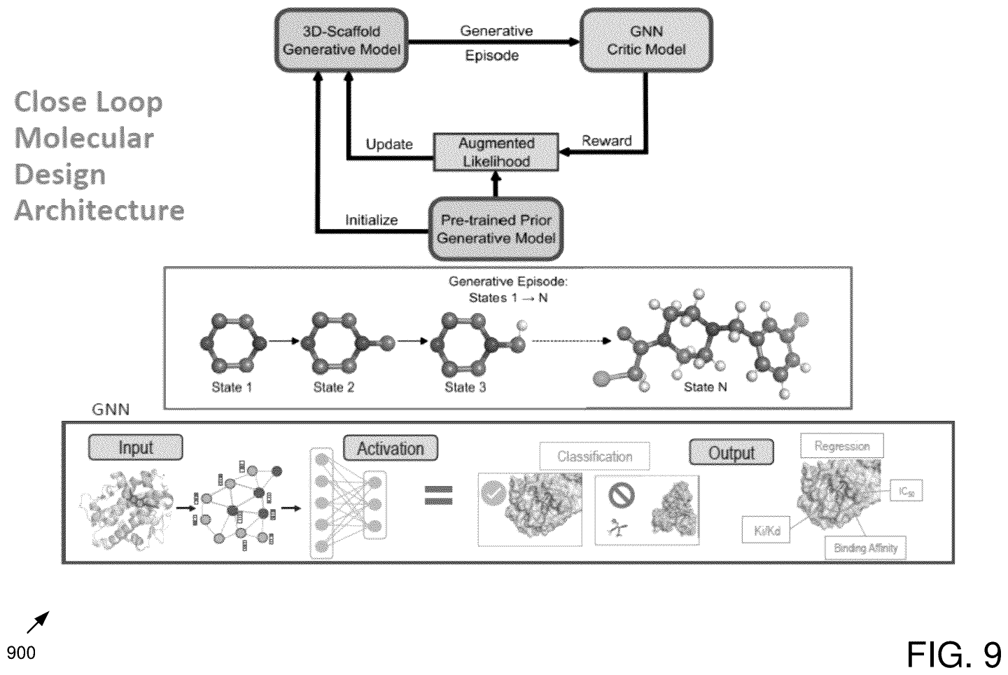

FIG. 9 is a block diagram of an alternative example architecture implementing the technologies described herein. In the example, a closed loop molecular design architecture 900 is shown. As shown, a pre-trained prior generative model can be used to initialize a three-dimensional generative model that generates a generative episode to the GNN critic model, which provides a reward to update the generative model.

In a generative episode, states 1 . . . N are presented. The GNN can process input and activation, resulting in classification, and output as shown.

### Example 25—Example Overview

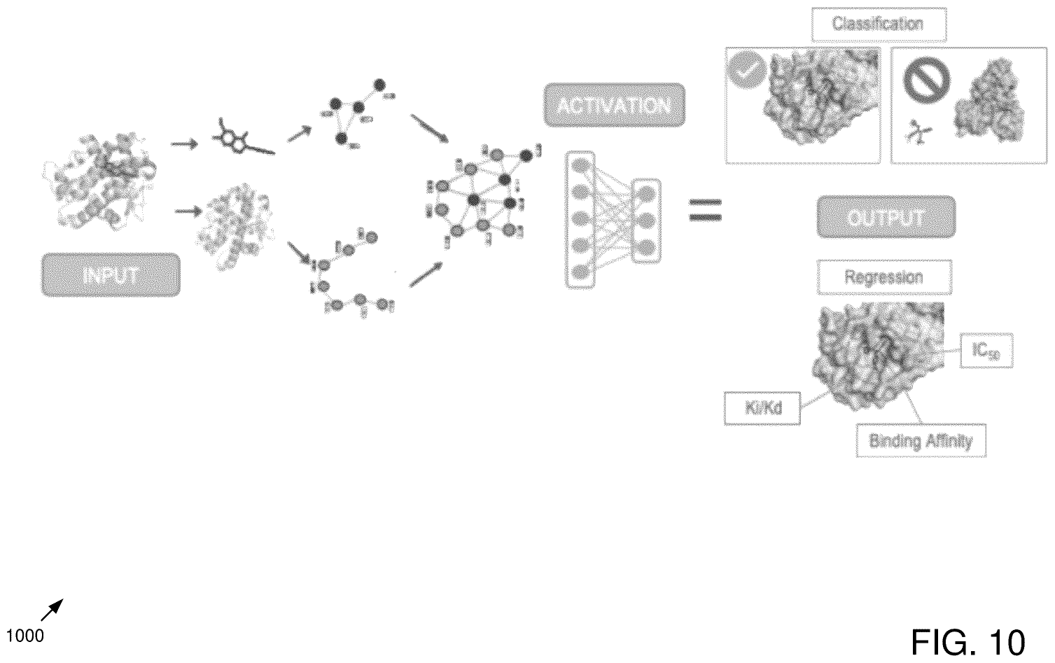

FIG. 10 is a block diagram of an overview of decoding protein interaction with domain-aware machine learning that can be used to implement the technologies described herein.

In the example, identifying protein interactions can help accelerate functional discovery and optimize new candidates. Graph neural networks (GNNs) can consider experimentally resolved three-dimensional atomic structures of both protein target and candidate molecules to learn about the protein's interactions for the biophysical properties.

With AlphaFold2 from DeepMind, the GNN model can take their predicted three-dimensional protein structures as an input to identify candidates for new and emerging pathogens.

GNNs can serve as an interpretable and explainable artificial intelligence (AI) tool for predicted activity, potency, and biophysical properties of lead candidates.

### Example 26—Example Implementation: Results (Model Performance)

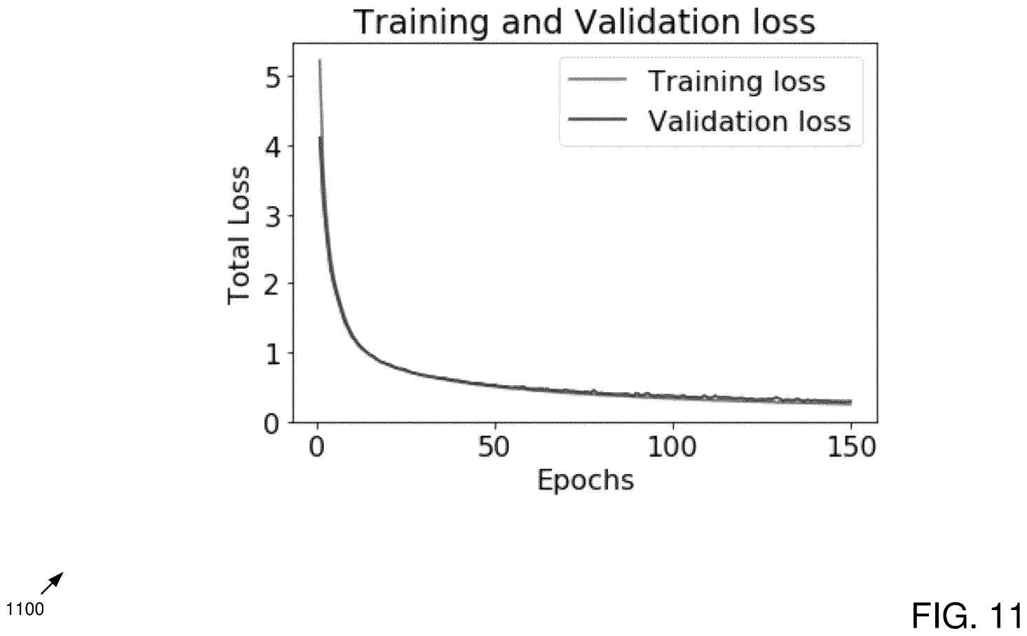

To ensure that the system is learning to build molecules based on the training data, the training and validation error over each epoch can be visualized. FIG. 11 is a graph of example training and validation loss, which represents the aggregated loss over the 150 epochs for the 3D-MolGNN_{RL} model. As loss decreases over time, it is seen that the model is able to effectively learn from the training data. This result is strictly evaluating the training and validation loss through the actor model to evaluate training performance, the drug-likeness scoring of the GNN model is discussed further in the next section.

### Example 27—Example Implementation: Results (Drug-Likeness Metrics for Generated Compounds)

The effectiveness of the reinforcement learning methods was investigated using different drug-likeliness metrics: quantitative estimate of drug-likeness (QED), water solubility (logS), synthetic accessibility (SA), and the octanol-water partition coefficient (logP). More information regarding the metrics is provided herein. A desirable drug candidate would score well in the metrics. Since the agent for the 3D-MolGNN_{RL} is a scaffold based generative model, the focus was on generating compounds with piperazine as the scaffold. To ensure that only valid molecules are compared, the total list of generated compounds were filtered based on a modified Lipinksi rule. The rule suggests that for a candidate to be an acceptable drug-like compound, there should be no more than 5 hydrogen bond donors, no more than 10 hydrogen bond acceptors, no more than 5 rotatable bonds, a molecular mass between 200 and 500 Dalton, an octanol-water partition coefficient less than 5, and finally at least one aromatic ring in the structure. Once filtered, approximately 100 top compounds per method were obtained.

### Example 28—Example Implementation: Results (Comparison to 2D Generative Model)

To show the advantages of using 3D-based generative modeling over conventional 2D-based generative models (SMILES-based), one can compare the 3D-MolGNN_{RL} model to that of PaccMann_{RL}. For better comparison of the two methods, one can utilize a slightly modified version of the architecture to include the GNN critic model instead of the default PaccMann critic. This allows one to have a metric of comparison between a 2D and 3D generative model. For purposes of discussion, the modified PaccMann_{RL} model will be referred to as PM-GNN_{RL}.

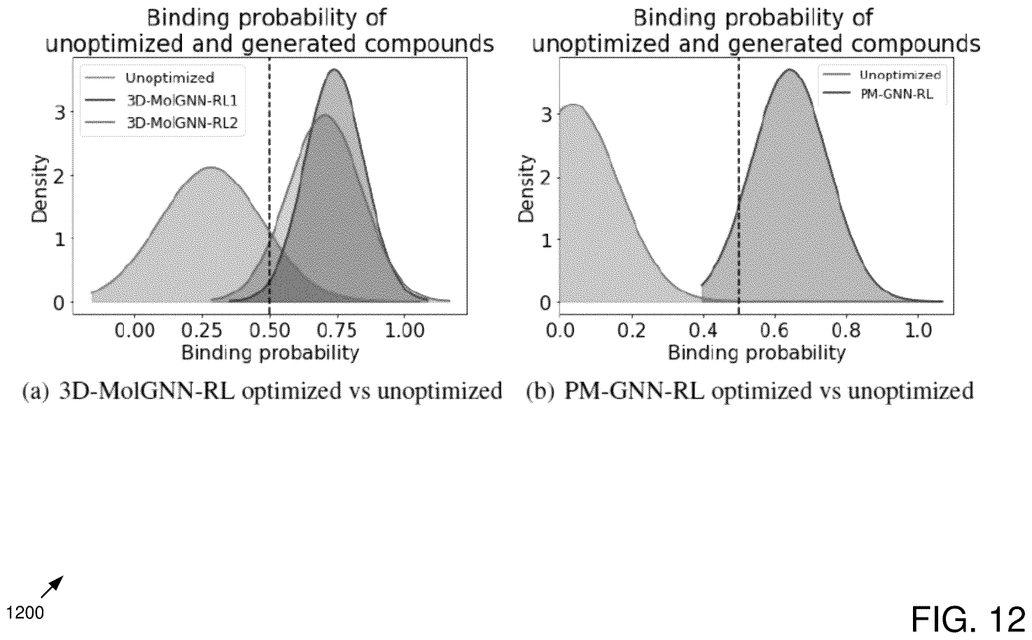

One goal was to demonstrate that the RL implementation of the methods was essential in producing molecules with a high binding probability. To accomplish this, molecules were generated for the protein target Mₚᵣₒ (PDB:6WQF), both with and without passing the reward from the critic back to the agent in each method. One can designate here the molecules generated without RL optimization as unoptimized. To fairly compare the optimized vs unoptimized methods, one can ensure that the unoptimized models were trained and tested on the same datasets used for training the respective RL models. The comparison of binding probabilities of the generated compounds towards the Mₚᵣₒ target from the 3D-MolGNN_{RL} and PM-GNN_{RL} is shown in FIG. 12A and 12B, respectively. From the figure, it is evident that the RL mechanism has improved both agent's performance in producing candidates with high binding probability. The unoptimized molecules generated by the agent in 3D-MolGNN_{RL} and PM-GNN_{RL} show very low binding probability towards Mₚᵣₒ as the predictions have a low mean probability. On the other hand, when using biased compounds during RL optimization, one sees a significant increase in high binding probability compounds, showing the effects of incorporating RL with the generative models.

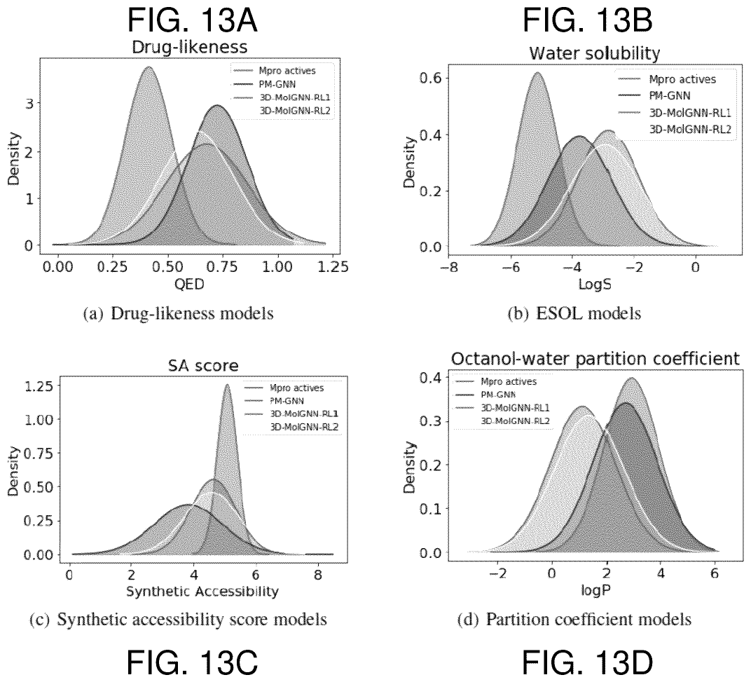

To further assess the novelty and properties of generated compounds, one can look at the four different drug-likeness properties listed above to serve as comparison metrics. FIG. 13 compares the metrics for both 3D-MolGNN_{RL} reward functions with known active molecules for Mₚᵣₒ. Further description of each metric is found herein. The first metric, QED, represents a quantification of the desirability of the drug (Bickerton et al., 2012). One can see that in FIG. 13A, the RL methods produce higher scoring molecules than the current experimentally determined Mₚᵣₒ actives with the mean of the PM-GNN_{RL} having a slightly higher increase in mean.

One can next look at the distributions of water solubility calculated using the ESOL method (Delaney, 2004). FIG. 13B shows that the methods score better than the current Mₚᵣₒ actives while also showing that the methods produce molecules with desirable solubility in water. The mean of the 3D-MolGNN_{RL}'s R1(s, t) model has the highest improvement relative to the mean of the Mₚᵣₒ actives while both the R2(s, t) and PM-GNN_{RL} models improve less.

The next metric, synthetic accessibility (SA), is a term included in each of the reward functions of the RL methods tested. FIG. 13C illustrates the distributions obtained by each method where a SA score closer to 1 is preferred. The two 3D-MolGNN_{RL} models have a consistent range of SA scores that center around 5, which indicates consistently above average scores. R1(s, t) shows an 8.78% improvement and R2(s, t) shows a 9.92% improvement. The PM-GNN_{RL} spans a much wider range of SA scores, but the mean is centered around 4. These three models only slightly edge out the existing Mₚᵣₒ actives.

The last metric is the octanol-water partition coefficient, or more simply known as logP. Looking at FIG. 13D, one can see how each method produces molecules spanning the range of −2 to 6. The 3D-MolGNN_{RL} model using the R1(s, t) reward yields a 61.21% improvement to the Mₚᵣₒ actives in logP value, whereas using the R2(s, t) reward yields a 53.08% improvement. In this metric, the PM-GNN_{RL} metric produces compounds very similar to the Mₚᵣₒ actives.

TABLE 1
Details about the drug-likeness metrics proposed in this work. One can
look at the 3D-MolGNN_{RL} model's two separate reward functions R₁(s, t) and R₂(s, t).
For each method, the top 3 candidates are chosen, and their metric scores listed.

| | QED | | | logS | | | SA | | | logP | | |
|---|---|---|---|---|---|---|---|---|---|---|---|---|
| Rank | Ist | 2nd | 3rd | 1st | 2nd | 3rd | Ist | 2nd | 3rd | 1st | 2nd | 3rd |
| 3D-MolGNN_{RL} R1 | 0.49 | 0.59 | 0.41 | −3.32 | −2.49 | −2.23 | 4.81 | 4.81 | 4.59 | 1.56 | 0.82 | −0.14 |
| 3D-MolGNN_{RL} R2 | 0.32 | 0.44 | 0.82 | −2.53 | −3.82 | −2.90 | 3.55 | 4.84 | 3.93 | 0.71 | 2.29 | 1.68 |
| PM-GNN_{RL} | 0.45 | 0.39 | 0.74 | −3.14 | −3.90 | −3.31 | 2.86 | 2.22 | 2.27 | 1.53 | 2.70 | 1.78 |

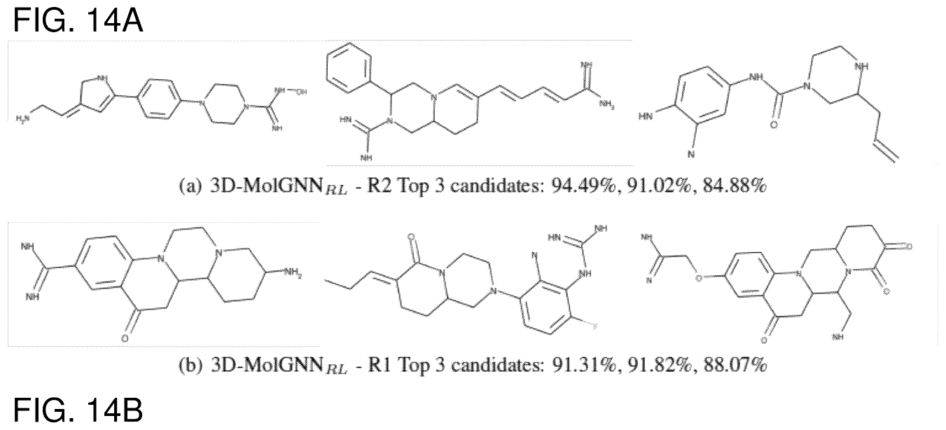

To compare how each method performed on a per-molecule basis, Table 1 gives the results for the top 3 candidates produced by each of the methods. These molecules were ranked based on their predicted binding probability with the Mₚᵣₒ target. One can see that molecules that are performing above average in one metric are not guaranteed to perform well in every metric. For example, the top candidates for each method are each above the mean SA for the given method, but produce below the mean for QED. 2D and 3D snapshots of the top 3 candidates from each method (R1 and R2) are shown as two-dimensional representations in FIGS. 14A and B. The candidates are shown in descending order from left to right. Their associated binding probability is listed in the subcaption also in descending order from left to right.

Since a goal is achieve de novo design, additional metrics such as validity, uniqueness, and novelty were considered among the piperazine-based generated compounds as shown in Table 2. Initial validity was determined by processing the molecules using cheminformatics tools. The methods of calculating these properties are described in further detail herein. From table 2, one finds that 3D-MolGNN_{RL} R1 and 3D-MolGNN_{RL} R2 produced greater than 95% valid compounds while also scoring close to 100% in uniqueness and novelty from the valid set. The PM-GNN_{RL} model, while not scoring quite as well in Uniqueness and Novelty, scores the highest in Validity. This shows that by incorporating more parameters into the multi-objective reward function, there is an improvement in the generation of novel drug candidates. Overall, 3D-MolGNN_{RL} R1 outperformed other methods by generating compounds with high Validity, Uniqueness and Novelty.

TABLE 2
Table outlining the three metrics used to evaluate the
compounds produced by varying reward functions and RL
models. Compounds were screened and scored for an overall
percentage based on validity, uniqueness, and novelty.

| Model | Validity | Uniqueness | Novelty |
|---|---|---|---|
| 3D-MolGNN_{RL} R1 | 99.9% | 100% | 99.9% |
| 3D-MolGNN_{RL} R2 | 97% | 99.9% | 99.9% |
| PM-GNN_{RL} | 100% | 93% | 93% |

### Example 29—Example Implementation: Results (Conclusions)

In the implementation, a new method was introduced to include both the 3D structure of protein target and the generated compounds to perform multi-objective lead optimization critical for drug design and discovery. It was demonstrated that the novel framework 3D-MolGNN_{RL}, which couples RL to a deep generative model based on a 3D-Scaffold, can generate target candidates built up atom by atom that are specific to a protein pocket. 3D-MolGNN_{RL} provides an efficient way to generate target specific candidates by learning to build molecules in 3D space while optimizing the binding affinity, potency, and synthetic accessibility. To accomplish this, one can utilize the protein for SARS-CoV-2 Mₚᵣₒ as a target for generating optimized inhibitor candidates. One finds that the model was able to generate molecules with better druglikeness, synthetic accessibility, water solubility, and hyrdophilicity than current Mₚᵣₒ active molecules. This was given by a >50% increase in QED, a >40% increase in solubility, a >8% improvement in SA, and a >50% improvement in hydrophilicity. One finds that the RL integration significantly improved the types of molecules generated by the untrained agents. It was demonstrated that by including more parameters into the multi-objective reward function, there is an improvement in generated novel target specific candidates. This gives confidence that our RL framework is effective at producing protein target specific hit candidates by leveraging the 3D structures of both the generated molecule and the protein pocket, a consideration not made by other molecular generation methods to date.

### Example 30—Example Performance Metrics (Quantitative Estimate of Druglikeness)

The QED metric represents a quantification of the desirability of the drug (Bickerton et al., 2012). The closer the score is to 1, the more desirable it is as a drug candidate.

The equation for QED is given as:

$$Q E D = exp ( \frac{1}{n} {\sum }_{i = 1}^{n} ln {d}_{i} ) ,$$

Where dᵢ is a series of desirability functions (d) belonging to eight widely used molecular descriptors. These are molecular weight (MW), the octanol-water partition coefficient (ALOGP), the number of hydrogen bond donors (HBD), the number of hydrogen bond acceptors (HBA), the molecular polar surface area (PSA), the number of rotatable bonds (ROTB), the number of aromatic rings (AROM), and the number of structural alerts (ALERTS).

The desirability function can be represented by a general asymmetric double sigmoidal function where d(x) is the desirability function for molecular descriptor x shown as:

$${d}_{i} ( x ) = {a}_{i} + \frac{{b}_{i}}{1 + exp ( - \frac{x - {c}_{i} + \frac{{d}_{i}}{2}}{{e}_{i}} )} \cdot [ 1 - \frac{1}{1 + exp ( - \frac{x - {c}_{i} + \frac{{d}_{i}}{2}}{{e}_{i}} )} ] ,$$

where ai, . . . , fi can be found in the supplementary table of the original publication (Bickerton et al., 2012).

### Example 31—Example Performance Metrics (Estimating Aqueous Solubility)

The metric, water solubility, calculates the log solubility (logS) of the molecule. In the technologies, the solubility can be determined by ESOL (Delaney, 2004). The majority of drugs possess a logS between −8 and −2. The more positive the value, the more water soluble the molecule.

ESOL as defined in Delaney (2004) can be calculated as the multiple linear regression of:
1. clogP
2. Molecular weight (MWT)
3. Rotatable bonds (RB)
4. Aromatic proportion (AP)
   given as:

Log(S_{w})=0.16−0.63clogP−0.0062MWT+0.066RB−0.74AP

### Example 32—Example Performance Metrics (Synthetic Accessibility)

Synthetic Accessibility (SA) is one of the most critical metrics to use in determining the simplicity in experimentally synthesizing a molecule. It need not be a score that dictates how effective the molecule is, but rather a practical measure of its complexity. The SA score is between 1 to 10, where 1 indicates an easily synthesizable molecule and 10 indicates a complex one.

The algorithm for calculating the SA score of a molecule (as represented in Ertl & Schuffenhauer (2009)) is given as:

SAscore=fragmentScore−complexityPenalty,

where the fragment score is calculated as a sum of contributions of all fragments in the molecule divided by the number of fragments in this molecule. The contribution of a fragment is obtained from a database of fragment contributions that were generated by statistical analysis of substructures in the PubChem collection.

The complexity penalty is a score given to characterize the presence of complex structural features in the molecules. It is defined as a combination of the following:

ringComplexityScore=log(nRingBridgeAtoms+1)+log(nSpiroAtoms+stereoComplexityScore=log(nStereoCenters+1) macrocyclePenalty=log(nMacrocycels+1) sizePenalty=nAtoms^{1.005}−nations

### Example 33—Example Performance Metrics (Hydrophilicity vs. Lipophilicity)

The hydrophilicity v. lipophilicity metric, known as logP, is the calculated octanol-water partition coefficient of a given molecule. The values represents if a drug is either very hydrophilic (−3) or very lipophilic (+10). This specific metric is present in Lipinski's rule as value that needs to be less than 5 to be considered a drug candidate.

To calculate the partition function for octanol-water partition coefficient that dictates whether a molecule is more hydrophilic or lipophilic one can utilize the RDKit package implementation of the Crippen approach (Wildman & Crippen, 1999). It simply calculates the sum of the contributions of each of the atoms in the molecule. Intramolecular interactions are accounted for my classifying atoms into different types based on their attached ai and neighboring atoms nᵢ:

$${P}_{calc} = {\sum }_{i} {n}_{i} {a}_{i} ,$$

where P can be further calculated into logP. A full list of the atomic descriptors and contributions can be found in the main text of Wildman & Crippen (1999).

### Example 34—Example Performance Metrics (Validity, Uniqueness, and Novelty)

To analyze the novelty of the compounds, one can look at how one calculates the validity and uniqueness of our compounds.

These are given as follows:

$$Validity = \frac{Number of valid molecules}{Number of generated molecules} ,$$

$$Unique = \frac{Number of unique molecules}{Number of valid molecules} ,$$

$$Novelty = \frac{Number of generated molecules not in training set}{Number of unique and valid generated molecules}$$

### Example 35—Example Top Candidates (3D Representations)

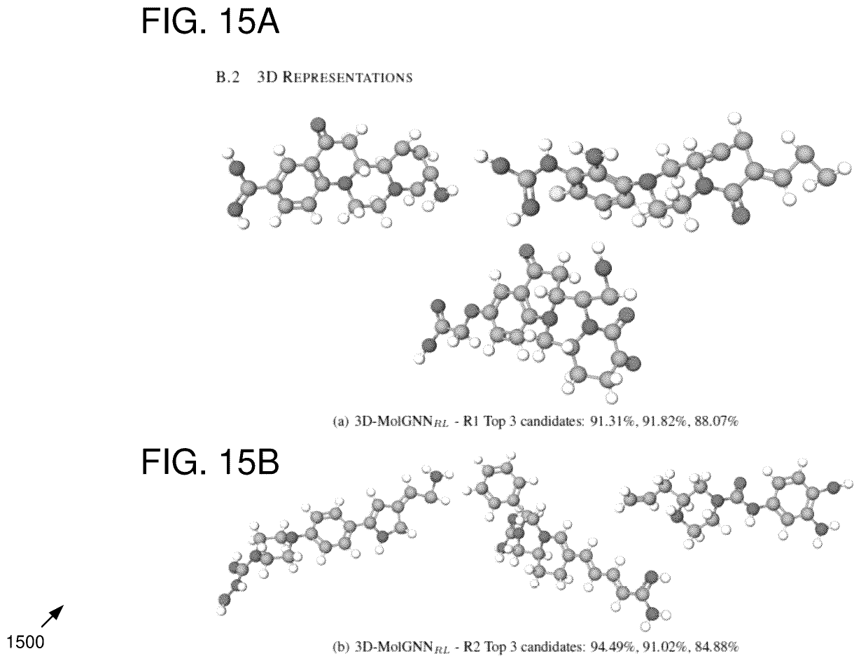

Three-dimensional representations of the top three (3) candidates for each reward function are shown in FIG. 15A and 15B. The candidates are shown in descending score order from left to right and down. Their associated binding probability is listed in the subcaption also in descending order from left to right.

### Example 35—Example Additional Properties

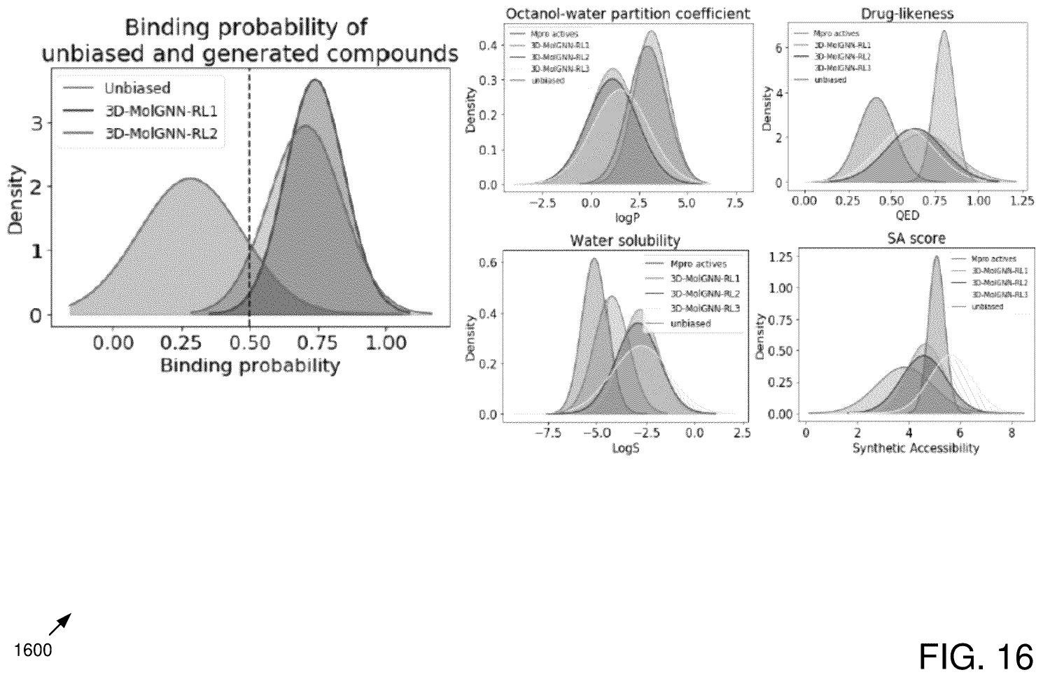

FIG. 16 is a graph comparing properties of molecules generated with the technologies described herein.

### Example 35—Example Ablation Study on Reward Function

To see the importance of optimizing for multiple properties in the reward function, one can test different training weight, and reward function combinations for the model. The most important 2 were listed herein, but 2 more reward functions were tested to test performance.

One can use the following combinations and compare how their output scores in the drug-like metrics. One sees a steady decrease in performance from Reward 1 to Reward 3 showing that the inclusion of Binding Probability is important for generating molecules with a drug-like features. One can also compare the 3D method with the 2D protein inclusion using the original PaccMann Method but with the GNN instead of their PaccMann critic to see if the 2D features have an effect.

R₁(sₜ)=α.C_{BP}(B,Cₜ)+β.C_{EA}(B,Cₜ)+(1−γ.C_{SA}(Cₜ))

R₂(sₜ)=α.C_{BP}(B,Cₜ)+(1−β.C_{SA}(Cₜ))

R₃(sₜ)=α.C_{BP}(B,Cₜ)+(1−β.C_{SA}(Cₜ))

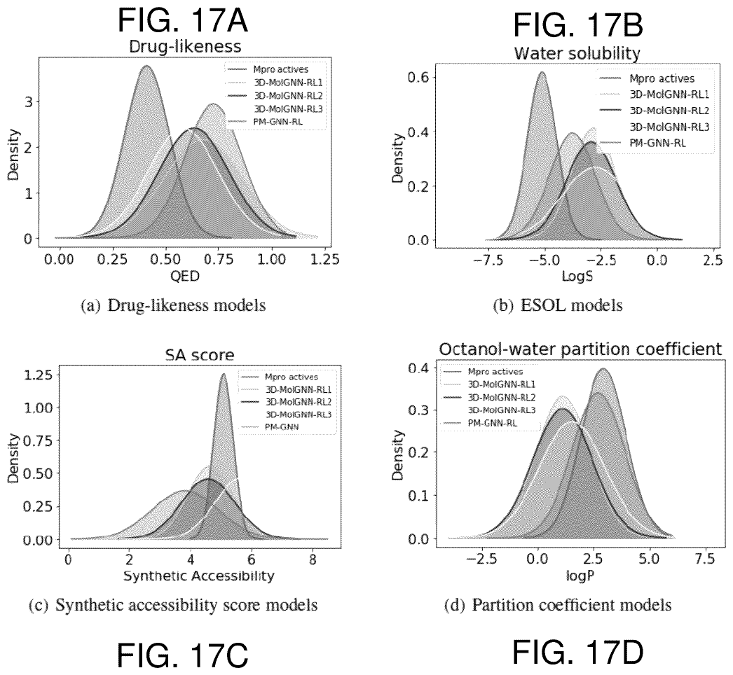

FIG. 17A-D are graphs comparing properties of molecules produced from the technologies described herein against experimentally identified active compounds for Mₚᵣₒ target. Overall, the drawings show that the complete three (3) term reward function for the 3D-MolGNNRL method produces the best scoring molecules overall for the given drug-like metrics.

### Example 35—3D-Scaffold Hyperparameters

For the 3D Scaffold model one can use the following hyperparameters: batch size of 2, split of 1000-500 training, validation set, 150 max epochs, learning rate of 0.0001, learning rate patience of 10, learning rate minimum of 1e-6, learning rate decay of 0.5, atom-wise representation layer size of 32, cutoff radius of local environment of 10, interaction layer size of 6, 25 gaussians to expand distances with a max distance of 15 and 300 distance bins. Other hyperparameters are possible.

### Example 36—GNN Hyperparameters

Hyperparameters such as network depth, layer dimension, and learning rate can have a large effect on model training and the weights in the final realized model. Therefore, one can perform a number of trainings to examine combinations of learning rate, number of attention heads, and layer dimension. One can use the parameters from the best model to create a baseline: a learning rate of 0.0001, four attention heads, and a layer dimension of 70 produced an average test AUROC of 0.854. The test parameters and top scoring configuration are outlined and highlighted in Table 3. The hyperparameters that performed best were a learning rate of 0.0001, two attention heads, and a dimension of 70. These parameters resulted in an average test AUROC of 0.864, still scoring lower than the implemented baseline configuration. In practice, other configurations are possible.

TABLE 3
Values of examined hyperparameters: learning rate (lr), number
(N) of attention heads, and dimension (D) of the GAT layer in
each attention head. A grid search was performed on each combination
of parameters. The optimal combination is shown in bold.

| lr | N | D |
|---|---|---|
| 0.001 | 2 | 70 |
| 0.0001 | 3 | 140 |
| 0.00001 | 4 | 210 |
| | | 280 |

Trainings were carried out over 200 epochs on a quarter of the data taken from our dataset consisting of 2,000 samples per target with 79 targets, using the same train-test split for all trainings The decreased performance compared to the models is expected due to the reduced set of data used to train the model. Thirty of the 36 combinations trained without error and are shown in Table 4.

TABLE 4
Values of examined hyperparameter set
with average train and Test ROC.

| HYPERPARAMETER SET | TRAIN ROC AVG | TEST ROC AVG |
|---|---|---|
| LR_0.001_N5_D70 | 0.754 | 0.771 |
| LR_0.001_N4_D210 | 0.606 | 0.647 |
| LR_0.001_N4_D140 | 0.605 | 0.661 |
| LR_0.001_N3_D70 | 0.834 | 0.835 |
| LR_0.001_N3_D280 | 0.610 | 0.630 |
| LR_0.001_N3_D210 | 0.672 | 0.694 |
| LR_0.001_N3_D140 | 0.714 | 0.742 |
| LR_0.001_N2_D70 | 0.937 | 0.861 |
| LR_0.001_N2_D140 | 0.788 | 0.781 |
| LR_0.001_N2_D280 | 0.762 | 0.755 |
| LR_0.001_N2_D210 | 0.767 | 0.773 |
| LR_0.0001_N4_D128 | 0.898 | 0.854 |
| LR_0.0001_N3_D70 | 0.894 | 0.855 |
| LR_0.0001_N3_D210 | 0.930 | 0.860 |
| LR_0.0001_N3_D140 | 0.917 | 0.862 |
| LR_0.0001_N2_D70 | 0.912 | 0.864 |
| LR_0.0001_N2_D210 | 0.950 | 0.861 |
| LR_0.0001_N2_D140 | 0.940 | 0.853 |
| LR_0.00001_N4_D70 | 0.707 | 0.726 |
| LR_0.00001_N4_D210 | 0.776 | 0.783 |
| LR_0.00001_N4_D140 | 0.748 | 0.769 |
| LR_0.00001_N3_D70 | 0.735 | 0.758 |
| LR_0.00001_N3_D210 | 0.778 | 0.772 |
| LR_0.00001_N3_D140 | 0.763 | 0.755 |
| LR_0.00001_N2_D70 | 0.713 | 0.749 |
| LR_0.00001_N2_D210 | 0.776 | 0.783 |
| LR_0.00001_N2_D140 | 0.774 | 0.782 |

### Example 37—Math Notation

The following math notation can be used.
Sₜ—state of the molecule at for action t
T—Terminal state/Max length of molecule
S—all possible states
Rₜ—reward for the partial molecule at step t
Mₜ—candidate molecule
B—target protein
X_{G}—Target embeddings from protein-VAE for SELFIES-VAE G—generator
C_{BP}—Critic for Binding Probability prediction
C_{SA}—Critic for SA score prediction
C_{EA}—Critic for Experimental Affinity prediction
Π(Θ)—optimization policy
N—steps per episode
P_{Θ}—probability associated with the action t
Zₙₑₓₜ—ground truth of the next atom
\hat{p}_{type}^{Z}^{ₙₑₓₜ}—probability that the model assigns to that type at the current step.
\hat{p}ⱼ^{b}—that the model assigns for the distance between rⱼ and rₙₑₓₜ to fall into distant bin b∈B at the current step

### PM-GNN_{RL} Model

Π(Θ)=Σₛ_{ₜ}_{∈S}P_{Θ}(sₜ)R(sₜ)

$$R ( {s}_{t} ) = \{ \begin{matrix}α . {C}_{BP} ( B , {C}_{t} ) + β . {C}_{SA} ( {C}_{t} ) , & t = T \\ 0 , & t < T\end{matrix}$$

### 3D-MolGNN-_{RL} Model

Π(Θ)=Σₛ_{ₜ}_{∈S}(P_{Θ}(sₜ)−R(sₜ))

P_{Θ}(sₜ)=P_{Θ}(sₜ)^{type}+P_{Θ}(sₜ)^{dist}

P_{Θ}(sₜ)^{type}=−log(p_{type}^{Z}^{ₙₑₓₜ})

P_{Θ}(sₜ)^{dist}=Σⱼ₌₁^{N}Σ_{b∈B}qⱼ^{b} log(\hat{p}ⱼ^{b})

R₁(sₜ)=α.C_{BP}(B,Cₜ)+β.C_{EA}(B,Cₜ)+(1−γ.C_{SA}(Cₜ))

R₂(sₜ)=α.C_{BP}(B,Cₜ)+(1−β.C_{SA}(Cₜ))

R₃(sₜ)=α.C_{BP}(B,Cₜ)+(1−β.C_{SA}(Cₜ))

### Example 36—Example Implementation in a SARS-CoV-2 (M^{pro}) Context (Introduction)

The following describes an AI-accelerated design of targeted covalent inhibitors for SARS-CoV-2 using the technologies described herein. The COVID-19 pandemic has claimed more than 6 million lives globally since it started in early 2020, and millions of people are suffering from long term COVID sequelae. The continuing emergence of new SARS-Cov-2 variants of concern coupled with their ability to spread globally underscore the need for streamlined pipelines for the discovery of novel antiviral treatments. The recent FDA approval of COVID-19 pharmacotherapeutics is a positive development in the ability to treat the disease post-infection; however, the potential for escape variants due to immune evasion and selection in immunocompromised individuals remains high. As such, discovery of antiviral drugs with high efficacy and safety to treat COVID-19—and other related viruses—remains exigent. As the typical timeline for discovery of novel drugs requires years to decades with conventional approaches, repurposing of existing drugs has been a major focus of discovery efforts. Therefore, methods that speed up and otherwise optimize the high throughput screening of novel potential candidates could greatly increase the chemical space of pharmacological modulators, and thus expand the opportunities to treat this disease—by both targeting current strains and efficiently finding leads that target new mutants as they arise.

There is immense promise in the design of inhibitors with various chemical warheads that efficiently inhibit the SARS-CoV-2 main protease (M^{pro}) and papain-like protease (PL^{pro}), but computational methods lack systematic tools—especially automated tools—for covalent inhibitor design. In particular, the viral cysteine protease M^{pro} has been verified as essential for viral replication; therefore, inhibition of this enzymatic activity could stall the viral lifecycle of SARS-CoV-2. The M^{pro} active site can form both covalent and non-covalent interactions with inhibitory ligands and is considered a desirable target in part because no homologous protein exists in humans, and a selective inhibitor would therefore have low toxicity.

A number of studies have focused on developing antiviral non-covalent inhibitors against M^{pro} based on high throughput virtual screening using a docking workflow. However, a design of covalent inhibitors has been less common for the SARS-CoV-2 M^{pro} due to the lack of mechanistic knowledge and electrophilic warhead design strategies. To this end, one could discover highly selective covalent inhibitors of M^{pro} through prioritizing noncovalent interactions with poorly conserved active site amino acid residues that position the molecule to react with a catalytic Cys145 residue in the active site (FIG. 18A). FIG. 18A shows a three-dimensional structure representation of SARS-CoV-2 main protease, M^{pro}, and its active site labeled with S1-S4 binding subsites interacting with one of covalent hit (compound C).

In seeking to apply the novel covalent ligand discovery approach based on a deep learning (DL) model to a SARS-CoV-2 protein target, one can examine several steps that play a pivotal role in infection and replication, which offer many opportunities for one to apply an automated covalent inhibitor design approach (FIG. 18B). FIG. 18B depicts a computational workflow for targeted design of warhead and high-throughput virtual warheads screening that includes the DL model (3D-scaffold) to generate warhead and core scaffold-based covalent candidates, followed by Automated Model Engine (AME) for covalent docking of the generated candidates with M^{pro} to identify hits to be characterized experimentally and simulations. With the rapid progression of artificial intelligence (AI) and computing architectures, synergistic approaches combining machine learning with computational modeling and bench experiments are being implemented to reduce the drug discovery timeline. High throughput virtual screening (HTVS) is a particularly promising starting point because it enables the screening of large archives of chemical compounds and evaluation of ligand poses for biological activity. Although the computational approaches have demonstrated great potential, one area in the drug discovery process that could still particularly benefit from improved virtual screening approaches is covalent inhibitor design and discovery. Investment in the development of covalent inhibitors has ballooned over the last decade, in part due to the FDA approval of several rationally designed irreversible inhibitors, such as Ibrutinib, Afatinib, and Osimertinib. Covalent inhibitors can target otherwise intractable targets with high selectivity and are less sensitive to active site mutations that confer drug resistance to non-covalent inhibitors. Typically, covalent inhibitors are designed by appending an electrophile to a known inhibitor that can bind proximally to a nucleophilic active site residue. Therefore, de novo development of a covalent inhibitor remains a rarity. The automated covalent inhibitor workflow (e.g., FIG. 18B) and proposed candidates have a protease recognition moiety in combination with a reactive electrophilic molecule, or so-called ‘warhead’, but the core inhibitor structures diverge from typical protease inhibitors, which are usually designed to mimic peptide cleavage sites in non-structural peptides (NSPs).

Herein are described efforts to discover covalent small molecule inhibitors targeting SARS-CoV-2 M^{pro} using the newly developed covalent inhibitor design workflow in combination with covalent docking that leverages possible warheads, AI-generated core scaffold-focused covalent candidate analysis, high throughput experimental screening, time dependent inhibition, Native mass spectrometry (MS), X-ray crystallography, and antiviral assay testing (FIG. 18B). Through the process, promising candidates in the library were screened based on their binding affinity and interactions, which indicate their potential effectiveness as protease inhibitors. Several potential candidates were identified and further tested experimentally using Native MS and FRET-based screening assays, both to demonstrate the viability of the approach and because the experimental data are a key to the successive iterations of optimizations. Due to its potential for fast identification of protein-inhibitor covalent conjugates, one can choose a MS approach as a primary component of experimental validation. The kinetic assay confirmed that the covalent interactions were indeed inhibitory. From the screen, four of the AI-generated compounds turned out to be hits with high inhibition activities and are confirmed to be covalently bound using time-dependent MS experiments. As a final demonstration, one can verify covalent inhibition by characterizing room-temperature X-ray crystal structures of M^{pro} in complex with these compounds, indicating the covalent bonds between them and Cys145 and demonstrating experimentally their modes of binding. In addition, one can carry out atomistic molecular dynamics (MD) simulations to characterize conformational flexibility and elucidate the mechanism by which these inhibitors interacted with and bound to the M^{pro} active site while altering the enzyme's overall conformational dynamics. Together, the strategy provides a scalable platform for the rapid discovery of viable covalent lead candidates not only targeting SARS-CoV-2 but also for other emerging threats.

### Example 36—Example M^{pro} Implementation Results: High Throughput Virtual Screening

High throughput virtual screening of warhead-based covalent library down selected the top 25 candidate inhibitors for M^{pro}. One of the possible ways that the virtual screening could benefit covalent inhibitor discovery is via prediction of the poses that the inhibitor can adopt in the active site, as inhibitor pose, and 3D geometry is key to warhead bond formation with nucleophilic active site residues. The approach addresses this by learning not only local atomistic properties in the deep learning (DL) models, but also spatial interactions when designing knowledge and rule-based new candidates. Solving the covalent interaction problem would provide increased access to the expansive diversity that these libraries promise, including inhibitor candidates with tunable reactive warheads such as acrylamides, esters, chloroacetamides, nitriles, disulfides, maleimides, ketones, and pyrolidines—all of which can be generated using deep learning models such as 3D-Scaffold.

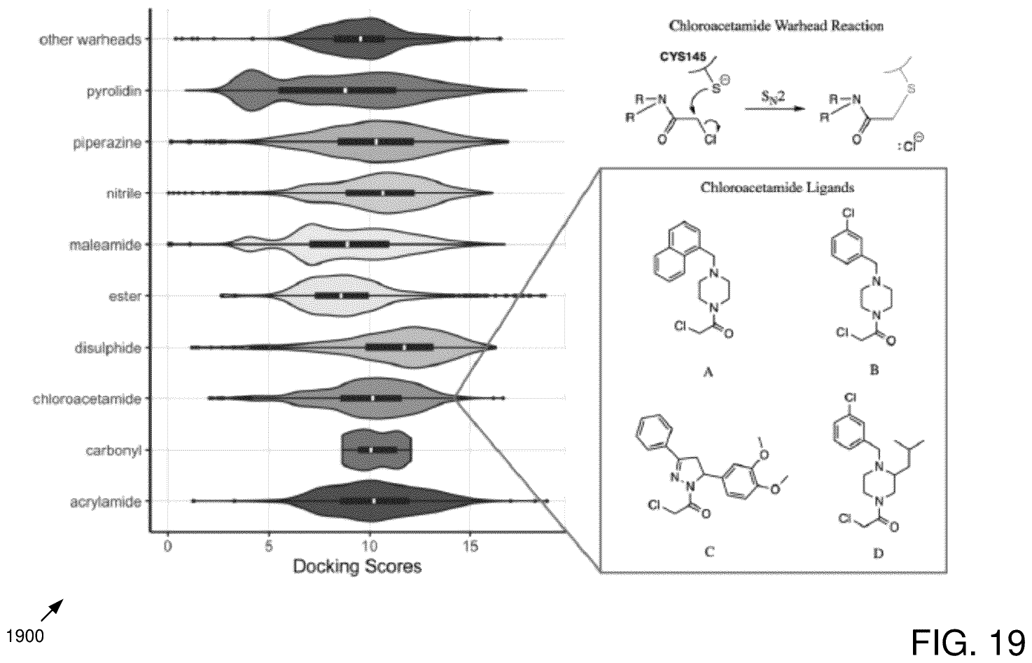

One can develop the Automated Modeling Engine (AME) for covalent docking that has the components needed to automate covalent docking of specified electrophilic warheads to a receptor Cys sidechain of the protein target (FIG. 18). One can initiate the inhibitor discovery process by using AME and a curated covalent antiviral library to perform iterative HTVS against M^{pro}. M^{pro} is a homodimer (PDB ID 6WQF), which features a cleft near the surface of each monomer, containing a catalytic dyad composed of a cysteine and histidine pair (Cys145 and His41). The screening library is comprised of compounds from multiple existing datasets and warhead-based compounds generated using the DL model 3D-Scaffold as described herein. Among the compounds are FDA approved drugs and candidates from the Enamine database (enamine.net) with 12 different scaffolds—namely acrylamides, ester, chloroacetamides, nitriles, disulfides, maleimides, and pyrolidines (FIG. 19). FIG. 19 shows violin plots 1900 of all AI-generated covalent compounds and curated databases grouped by warhead and core scaffold (piperazine and pyrrolidine) with distribution of the docking score against the M^{pro} target, where the higher values imply more favorable covalent binding. The electrophilic addition to Cys145 is shown for the chloroacetamide warhead (compound A, B, C, and D) potential hits for the covalent workflow.

All compounds contain a warhead functional group intended to bind covalently to Cys145 residue of M^{pro} via the nucleophilic thiol (SH) group. The use of 3D-Scaffold for molecular generation allowed one to generate thousands of compounds comprising 7 warheads to be used in the subsequent the molecular docking screening steps. 3D-Scaffold utilizes core scaffold (such as piperazine and pyrolidine) to constrain on the core scaffold and generate new candidates that typically have better performance than the parent candidates. This lowers the number of compounds to be screened rather than screening millions of molecules from various databases thereby reducing the computation cost and time.

The covalent docking workflow began with an extensive sampling of the ligands followed by an in silico covalent bond formation between the warhead of each ligand and Cys145. To rank the covalently bound candidates, the workflow uses a docking score and predicted binding affinity of the candidates. The candidates are ranked based on the calculated score (kcal/mol), which is a function of various protein-ligand interactions including van der Waals, electrostatic, hydrogen bonds, and desolvation contributions. The candidates were then ranked to those with the highest predicted binding affinity, binding pose, and most favorable interactions with M^{pro}. Based on the AI-assisted covalent modeling iterative design, one can finalize and select for experimental screening top 40 compounds (acrylamide, ester, chloroactamide warheads) from orderable databases such as Mcule (https://mcule.com/) and Enamine (https://enamine.net/). However, 10 of these compounds were not available to order. Of the 30 compounds available for purchase, 5 were excluded due to designation as pan-assay interference compounds (PAINS) based on the substructure filters of Baell and Holloway. These selected compounds were tested with various electrophilic warheads and compounds finalized for validation using experimental characterization techniques as discussed below.

### Example 37—Example M^{pro} Implementation Results: Fast Experimental Screening

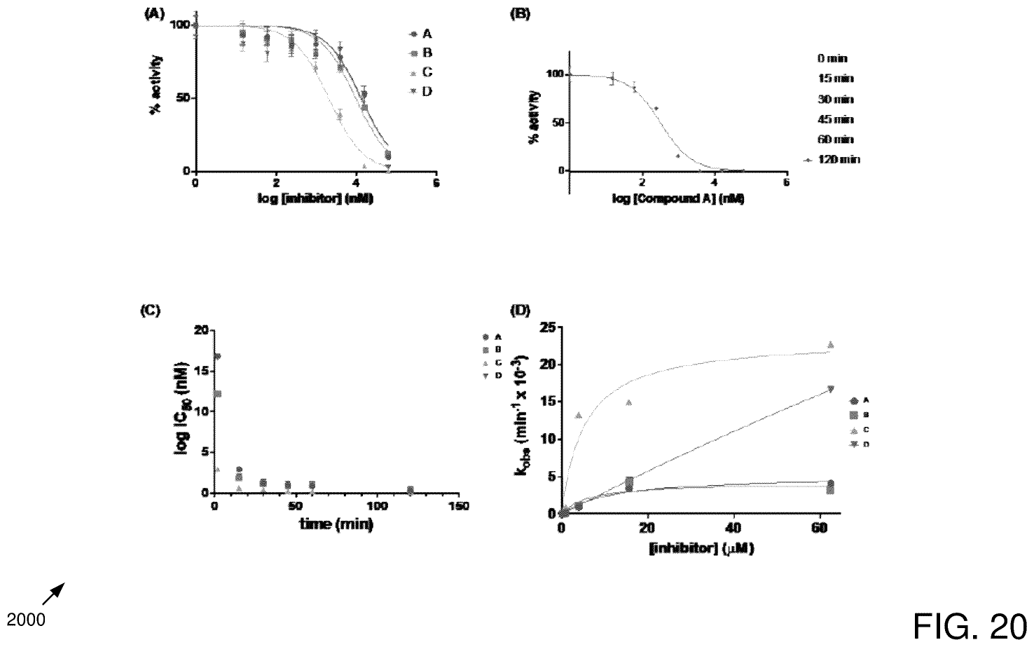

Fast experimental screen of candidate compounds validated a top 4 hits. To validate the computational leads, the remaining compounds were subjected to a primary SARS-CoV-2 M^{pro} activity inhibition screen in which they were pre-incubated with the enzyme and the initial velocities of cleavage of a FRET peptide were determined. The Z′-factor of the assay was 0.65, and the distribution of z-scores of compounds and positive (no inhibitor) and negative (no enzyme) controls is shown in FIG. 20.

FIG. 20 shows biochemical characterization of the kinetics of M^{pro} inhibition for screening hits using FRET activity assay. (A) IC50 curves for all compounds after 2 min of co-incubation of enzymes and inhibitors. (B) Representative time-dependent IC₅₀ plot for compound A. (C) Assessment of time dependence of IC₅₀ values for all compounds. (D) Saturation profiles for each inhibitor.

At least 25% inhibition was observed for seven compounds, with four resulting (Compounds A-D, FIG. 20) in the lowest residual activity at 20 μM (Table S1) that became the focus. Among the four hits, compound C has the highest binding score that was predicted to be stabilized by an interaction between the two ethers with Asn142. One can also note from the computational analysis that the highest binding score does not always leads to high affinity.

After the qualitative screening of the compounds, one can perform quantitative FRET-based inhibition assays for compounds A-D. All four compounds (A-D) demonstrated dose-dependent enzyme inhibition, with micromolar IC₅₀ₛ (FIG. 20A). Compound C showed the lowest IC₅₀ in the preliminary analysis (2.39±0.09 μM), which was at least 5-fold lower than the IC₅₀ₛ of the other compounds. To investigate the possibility of covalent inhibition for the screen hits, one can determine the impact of pre-incubation of enzyme and inhibitor using the inhibition assay, with the expectation that any covalent inhibitor should demonstrate a decrease in IC₅₀ with increasing pre-incubation time. It was observed that pre-incubation of the inhibitors with enzyme resulted in a decrease in IC₅₀ for all four compounds, which is consistent with a covalent inhibition mechanism (FIGS. 20B and C). The most potent inhibitor, Compound C, had an IC50 of 160 nM after a 2-hour preincubation, which is a 15-fold decrease from the initial IC50 measurement (t=2 minutes). Although compounds A, B, and D showed weaker final inhibition, they showed a 54-, 32-, and 39-fold decrease in IC50, respectively, when comparing the initial and final inhibition values.

TABLE R1
IC₅₀, K_{I} and k_{inact} values for M^{pro} screening hits.
IC₅₀ values are reported with standard deviation. K_{I} and
k_{inact} values are reported with SEM.

| | A | B | C | D |
|---|---|---|---|---|
| IC₅₀ (μM) - | 16.3 ± 1.3 | 12.8 ± 5.1 | 2.4 ± 0.1 | 16.0 ± 0.9 |
| 2 min | | | | |
| IC₅₀ (μM) - | 0.30 ± 0.03 | 0.40 ± 0.13 | 0.16 ± 0.03 | 0.41 ± 0.06 |
| 120 min | | | | |
| K_{I} (μM) | 11.5 ± 3.2 | 6.0 ± 4.0 | 5.3 ± 2.1 | ≥62.50 |
| k_{inact} | 5.1 ± 0.5 | 4.2 ± 0.8 | 23.5 ± 2.6 | ≥16.6 |
| (10⁻³ s⁻¹) | | | | |
| k_{inact}/K_{I} | 441 ± 297 | 697 ± 697 | 4,460 ± 417 | — |
| (M⁻¹ s⁻¹) | | | | |

While these IC₅₀ data indicate a covalent inhibition mechanism, true inhibition constants cannot be directly derived from these values because the observed enzyme inhibition is the result of a mix of covalent and non-covalent interactions. Therefore, in order to determine true inhibition constants for the inhibitors, a “saturation profile” analysis was conducted that allows for determination of K_{I} and k_{inact} through examining the decrease in M^{pro} activity over time for each respective inhibitor concentration (Table R1). The curve for compound D did not reach a plateau, and therefore did not display the desired saturation kinetics with the concentration range that was chosen for the experiment (FIG. 20D), so K_{I} and k_{inact} could not be determined for this inhibitor. However, the remaining compounds A-C showed the expected saturation kinetics and the analysis revealed low micromolar K_{Is} for M^{pro}. Further analysis of the same saturation plots additionally showed that compound C has the highest inactivation efficiency (k_{inact}/K_{I}) for M^{pro} (4,460 M⁻¹ s⁻¹), which is at least 4-fold higher relative to the other compounds values.

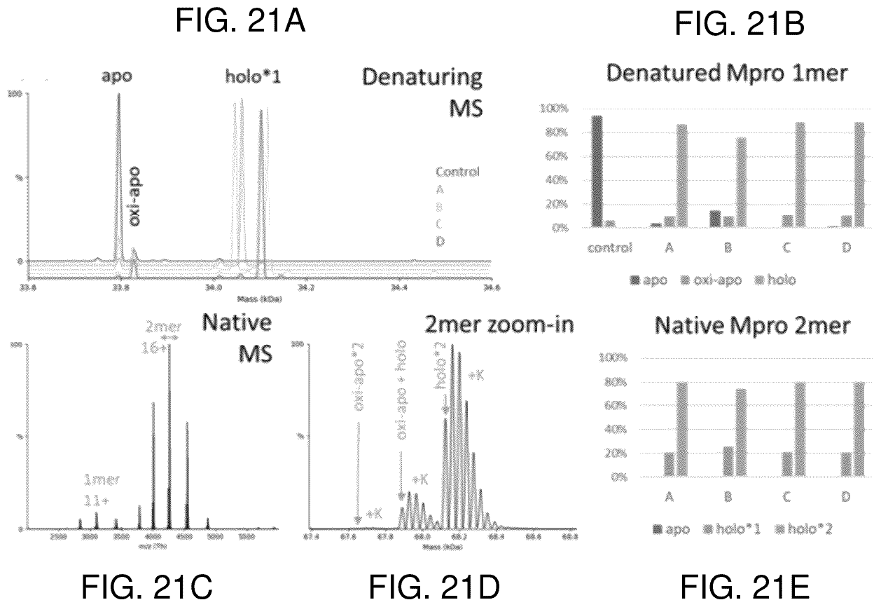

To further confirm the mechanism of action, MS was performed, which can unambiguously identify the protein-inhibitor covalent adduct formation. Analysis was conducted using intact, purified recombinant M^{pro} to confirm covalent binding of the four discovered compounds. After mixing M^{pro} with each of the compounds for 30 min, each reaction was then subjected to electrospray MS detection under either denaturing or native conditions. In denaturing MS (FIG. 21A), clear mass shifts indicated binding of the compounds. Treatment with all four compounds resulted in almost complete conversion of the apo M^{pro} to the respective inhibitor-conjugated forms, but addition of compound C resulted in the highest amount of the protein-inhibitor complex, which is in good agreement with the kinetic assay characterization. Because the addition of organic solvents may perturb the three-dimensional protein structure and eliminate noncovalent interactions, the denaturing MS results strongly support covalent binding. In addition, the mass shifts corresponded exactly to the mass of the protein-compound conjugates minus HCl as the leaving group from the chloroacetamide warhead, which is as expected from the designed chemical reactivity with the nucleophilic cysteine sidechain. Interestingly, we saw exclusively 1:1 binding per M^{pro} monomer (i.e., no significant peak at ˜34.4 kDa for 1:2 binding in FIG. 21A), suggesting binding to a single site on the protein. Moreover, a small percentage of oxidized M^{pro} (˜10%) was detected, which did not bind to the compounds. Cys145 is known to be susceptible to oxidize to the peroxysulfenic acid oxidation state. Upon oxidation of Cys145, the nucleophilic sidechain can no longer react with the electrophilic warhead, implying that the compounds conjugate to Cys145. A qualitative estimation of the different species (FIG. 21B) showed ˜80% M^{pro} bound to the designed compounds, suggesting the high efficiency of reaction in vitro.

The native MS analysis provided additional information on noncovalent interactions including protein higher-order structure and potential binding of reaction byproducts. Similar spectra were observed as reported previously by El-Baba et al. A representative native MS spectrum of M^{pro} mixed with compound A is shown in FIG. 21C, with most M^{pro} existing as homo-dimers, which is consistent with the stoichiometry of its known active form (all spectra in FIG. 21). The binding of the compound did not disrupt the dimers significantly, suggesting the inhibition was achieved via blocking of active site instead of perturbation of protein higher-order structure. FIG. 21D shows the mass distribution of the dimers. Despite the significant amount of nonspecific adducts (multiple potassium ions), the mass of the fully protonated peaks can be confidently assigned (peaks labeled with green arrows). Again, the holo Mpro dimer mass increased from the apo dimer by the mass of the compound minus HCl. This mass shift is in agreement with the denaturing MS data, and also suggests the HCl was not associated with the protein and likely diffused into the bulk solution after 30 min incubation. All four compounds showed similar native MS spectra to compound A, and ˜80% of the dimers were fully occupied by the compounds (FIG. 21E). 20% of the dimer had mixed protomers with one holo and one oxidized apo. Negligible (<1%) amounts of oxidized apo dimer were seen.

FIG. 21A shows intact mass measurement of M^{pro} after binding to the designed compounds under denaturing conditions. One compound was covalently linked to each M^{pro} monomer. A small percentage of M^{pro} were oxidized (“oxi-apo,” deoxidation on Cys, +32 Da). FIG. 21B shows percent of apo, oxidized apo, and holo M^{pro} estimated from the denaturing MS data. FIG. 21C shows native MS of M^{pro} mixed with compound A. M^{pro} existed primarily as dimer under this condition. FIG. 21D shows a zoom-in of the deconvoluted mass distribution of M^{pro} dimer mixed with compound A, with main species labeled by the arrow. The densely spaced peaks right to the labeled peaks are from multiple nonspecific adducts of potassium (+K). Most dimers were linked to two compounds (holo*2), but ˜20% of the dimers had one oxidized apo monomer and holo monomer. FIG. 21E shows percentage of apo, holo*1 (with one monomer bound), and holo*2 (both monomers occupied) of the Mpro dimer based on the native MS data. The apo dimers were <1% and nearly undetectable.

### Example 37—Example M^{pro} Implementation Results (Antiviral Activity of the Four Hits)

The in vitro antiviral activity of all four hits was evaluated in Vero E6 (CRL-1586) cells. The antiviral properties of the inhibitors were assessed in the absence and presence of an efflux pump inhibitor (CP-100356, labeled PZ). The cytotoxic effect of Compound A+PZ is less than other compounds. The cytotoxic effects for two test compounds Compound B+PZ and Compound C+PZ at 8.33 μM and higher concentrations are too high to show protective effect, because viability rate of cells infected by virus and treated with these compounds at high doses are even lower than the viability rate of cells infected by virus without drug treatment. Because of high cytotoxic effects of these compounds at high doses, the high doses were removed in the EC50 curve fit for these compounds. Unfortunately, cytotoxicity was detected for all four inhibitors at concentrations up to 10 μM (FIG. S11), but the mechanism of action was additionally characterized for these compounds to serve as proof of principle to demonstrate that AI-assisted workflows can lead to rapid discovery of covalent inhibitors.

### Example 38—Example M^{pro} Implementation Results: High resolution X-ray Crystal Structure of M^{pro}

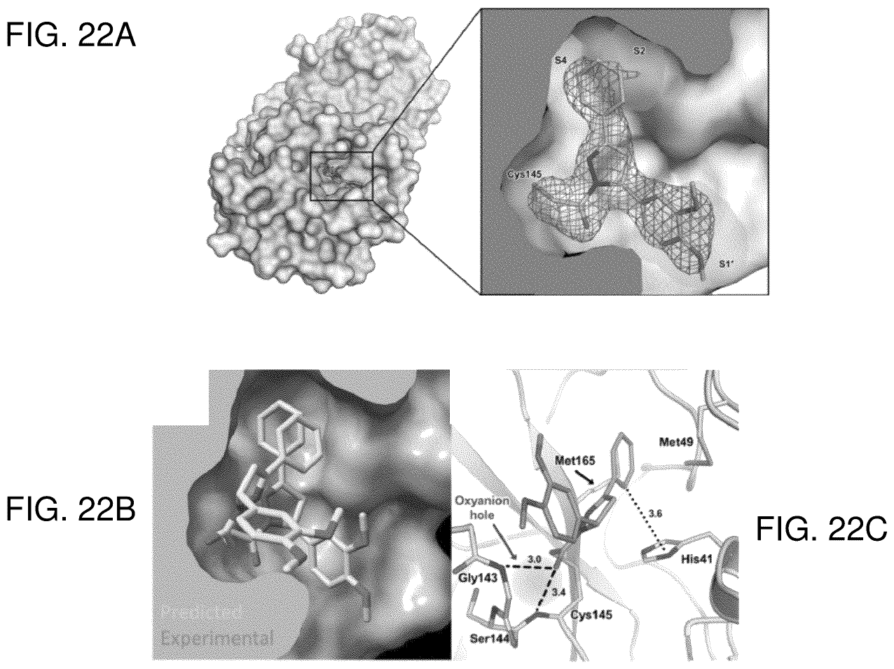

The high-resolution X-ray crystal structure of M^{pro} in complex with chloroacetamide hits defined the detailed mechanism of action. The kinetic and MS characterizations of the M^{pro} screen hits are in good agreement with a covalent mechanism of action. To define the specific site and the mode of inhibitor binding, one can obtain room-temperature co-crystal structures of compounds A, C, and D bound to M^{pro} at 1.9-2.0 Å resolutions. Each ligand was modeled into unambiguous electron density and the predicted poses from the virtual screen juxtaposed with the experimentally co-crystallized ligands are also depicted (FIG. 22A-C). The critical covalent interaction with Cys145 was found by the docking simulation. The covalent bonds between the catalytic Cys145 and chloroacetamide warheads of each ligand were confirmed in the crystal structures to form a thioether linkage after Cl⁻ leaves. The carbonyl groups of each compound are directed into the oxyanion hole and make hydrogen bonding interactions with the main-chain amide NH groups of Gly143 and Cys145. Compounds A and D insert deeper into the oxyanion hole, with the hydrogen bond distances of 2.7-2.8 Å with Gly143 and 3.0-3.2 Å with Cys145, whereas these hydrogen bonds are longer for compound C, with the distances of 3.0 Å and 3.4 Å, respectively.

The aromatic P2 substituents of the studied ligands (phenyl of C, naphthyl of A, and chlorobenzene of D) extend into the S2 subsite and are directed towards Met165 of the S4 subsite. S2 and S4 subsites are guarded by Met49, and by Met165 and Leu167, respectively, and are not fully open in inhibitor-free M^{pro} structures; therefore, these subsites are cryptic as their full volumes are blocked by the side chains of these residues. In peptide substrates and peptidomimetic inhibitors, large hydrophobic P2 and P4 groups carve out the S2 and S4 subsites by sterically driving these residues away from their positions in unbound enzyme. The current studied ligands lack large enough substituents to evict Met49, Met165 or Leu167 to fully extend into the S2 and S4 subsites. Instead, the P2 substituents are positioned between Met49 and Met165 and are oriented almost perpendicular to the imidazole ring of the catalytic His41 (FIG. 22). The P2 naphthyl of A makes the closest C—H . . . π interactions with His41 of 3.3 Å, whereas the P2 phenyl of C is father away (˜3.6-3.7 Å). Due to Cl substitution, the P2 group of D shifts even farther up from His41, with the distances of ˜4.5-4.6 Å, indicating no significant interactions with the imidazole π-system. Rather, the Cl substituent is 3.4 Å away from the sulfur of Met165, making perhaps a favorable interaction. In addition to the P2 groups, compounds C and D also harbor P1′ groups, 2,3-dimethoxybenzene and isobutyl, respectively, that are directed into the shallow S1′ subsite. 2,3-Dimethoxybenzene is stacked against the Asn142 side chain with the closest distances of 3.6 Å, whereas the isobutyl group forms no interactions with the M^{pro} active site residues, which is manifested by a weak electron density for this substituent.

FIG. 22 Room-temperature X-ray crystal structure of SARS-CoV-2 M^{pro} in complex with compound C (PDB ID: 8DLB) and comparison with the docked structures. FIG. 22A shows a single subunit of Mpro shown with predicted binding pose of compound C and binding subsites are labeled S1-S4. FIG. 22B shows a sectional view of the binding pocket occupied by the predicted ligand pose superimposed with the experimentally determined crystal structure. FIG. 22C shows polar contacts between the binding pocket residues and compound C.

### Example 39—Example M^{pro} Implementation Results: MD Simulations

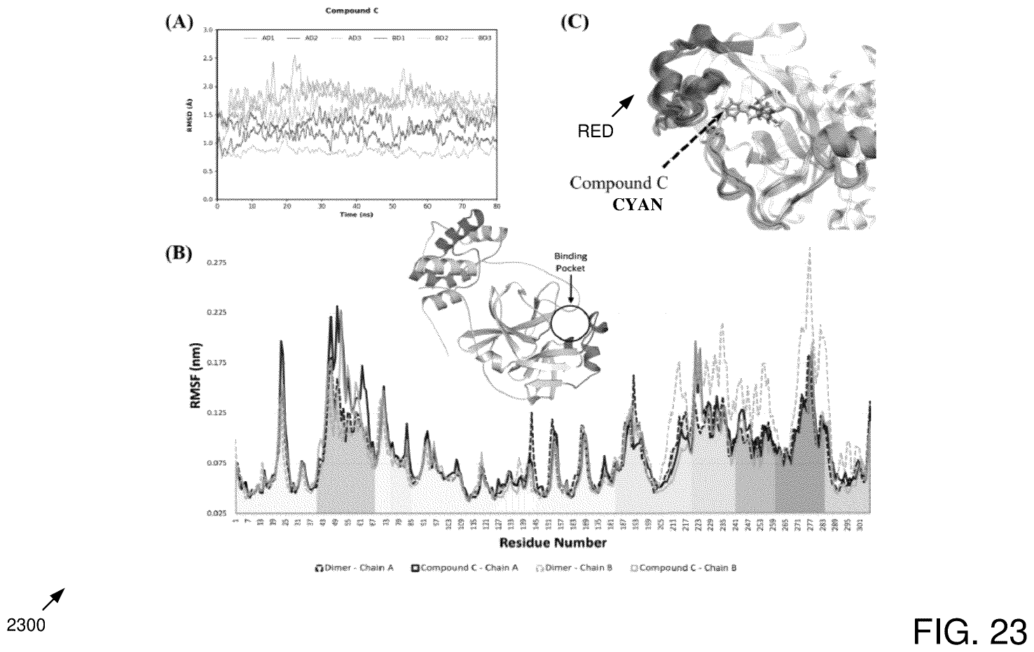

MD simulations revealed potential conformational dynamics for further hit optimization. To elucidate the dynamics and conformational flexibility underlying our computationally screened and experimentally validated hits, MD simulations of M^{pro} complexes with the four compounds were carried out (FIG. 19). Of particular interest was understanding conformational modulation of the M^{pro}/Compound C complex, so it was compared to the protein in its apo-state. The M^{pro} dimer consists of two chains (A and B) which are comprised of three subdomains (for residues ranging from 8-101 (D1), 102-184 (D2) and 201-303 (D3)). Domains D1 and D2 are essential for catalytic activity, whereas domain D3 is responsible for the dimerization and plays a key role in protease activity. The Native MS results demonstrated that >80% of dimers had their both active sites occupied by an inhibitor, however, it was desired to investigate computationally which structural regions were most affected by the bound ligand (compound C) in the active site of one of the chains of the M^{pro} dimer. The procedure described herein was followed to simulate the M^{pro} dimer in its apo and complexed states. Root mean square deviation (RMSD) analysis was performed of chains A and B and their respective domains to identify conformational dynamics within the protein. To determine the contribution of individual amino acids to the structural changes, the root mean square fluctuations (RMSF) were analyzed at the Cα atom of every residue. The RMSD plots (FIG. 23A) for the various domains show that binding of compound C in the active site of M^{pro} results in higher fluctuations in the D1 domain of chain A, followed by the D3 domains of chain A and chain B. This implies that binding of the ligand in the catalytic domain of chain A resulted in allosteric conformational changes in D3 domain of chain B. FIG. 23B highlights the RMSF across distinct regions of the M^{pro} both with and without ligands bound where, as expected, ligands with higher affinity can stabilize the protein conformation to a greater degree.

In addition, conformational differences between the two simulations occur at most helical regions (exceptions being the yellow/orange beta sheet and interdomain regions), with the largest discrepancy at the monomer region closest to the ligand binding pocket. At the S2 helix (red: FIG. 23C) in the D1 domain of chain A (residues 40-67), the Met49 residue shows higher fluctuations which results in the opening of the S2 subsite to accommodate the ligand. Conversely, His41 shows lower fluctuations upon binding of the ligand in the pocket (FIG. 23) suggesting that binding of the ligand in the active site stabilizes the His41 residue by interacting with the ligand as observed in FIG. 22C. Ligand binding also stabilizes the regions labeled orange and yellow, which are closer to the active site. Chain B was the most variable, especially at the N-terminal region (red) in ligand-bound state and C-terminal region (BD3 domain: green to purple) in ligand-free state indicating that binding of the ligand stabilizes the dimer. With ligands bound, chain A of the dimer had higher fluctuations than chain B mainly at the binding site. Similar to RMSD analysis, this implies binding of the ligands results in conformational changes in the protein in order to accommodate the ligand binding in its subsites (FIG. 22A). Similar RMSF analyses were done for the other three compounds as well and are included in the Supporting Information.

In order to observe the type of fluctuations from the RMSD and RMSF analysis and to identify the conformational dynamics occurring in the protein in ligand-bound and ligand-free states, the principal component analysis (PCA) was then run. PCA considers each frame of the simulation as an eigenvector and projects it on the initial structure using a covariance matrix. The first three PCs were obtained and the collective motions in the protein in various states were discerned. In the ligand-free state, PC1 and PC2 correspond to rocking motions, which are mainly specific to the D3 domains of both chain A and B which belong to the interface. These motions indicate the Ser1 and Glu166 interaction, which is important for dimerization. In the ligand-bound state, the PC1 and PC2 twisting motions are distinct from the ligand-free state. These twisting motions include mainly the D1 and D2 domains in both chains, which suggests that binding of the ligand results in the conformational changes in the entire protein. In this state, the ligand binding region shows higher fluctuations, primarily in the S1, S2, and S4 subsites of the binding site. From our dynamic cross-correlation (DCC Map or DCCM) analysis, we observed that subsite S2 (residues 40- 67) shows positive correlation with the subsites S1 and S4 in apo state, whereas shows opposite behavior in complex state. These conformational changes indicate the opening of the subsites, thus assisting the ligand into the binding site.

FIG. 23 shows conformational changes observed upon ligand binding in the active site of M^{pro}. FIG. 23A shows RMS deviation of various domains (D1, D2, and D3) in chains A and B. FIG. 23B shows RMS fluctuations in both chains of M^{pro} while being complexed with compound C reveal differences to dimer activity without ligands, highlighted by the protein region. FIG. 23C shows visual representation of RMSF fluctuations of compound C (cyan) bound in the active site of M^{pro} (color coded to regions/residues in the RMSF plot).

### Example 40—Example M^{pro} Implementation: Discussion

The chemical space search and HTVS involve a detailed molecular-level understanding of the role of the individual proteins, residues, and ligands involved, which helps shortlist potential compounds for testing in cell culture experiments using in vitro tools, which tend to be time- and labor-intensive and may otherwise be prohibitively expensive to use to assess all compounds. With the technologies described herein, a goal is to leverage an integrated approach that uses computational strategies including scaffold-based DL models for warhead candidate design that can be optimized iteratively based on experimental feedback. Such a study lead to the creation of a covalent-focused automated computational pipeline, a feature that distinguishes it from existing experimentally validated AI-driven M^{pro} HTVS approaches. In particular, a focus was on developing AI tools accelerating de novo design and discovery of novel compounds with a balanced library using 7 different scaffolds. A wide variety of computational protocols were used for HTVS, from simulations to covalent docking to screening the compounds against SARS-CoV-2 M^{pro}. Several groups have used docking as their primary screening criterion for a large combinatorial library to identify M^{pro} hits. A distinctive approach was followed; instead of screening a library of compounds, a custom electrophilic warhead library was generated with the 3D-scaffold DL model using the core scaffolds obtained from the experimentally validated inhibitors thus generating valid and synthesizable compounds for computational and experimental testing. The generated library of compounds was then screened with the automated covalent docking pipeline to make a covalent bond with Cys145 and optimize interactions within the pocket. The compounds were ranked based on their docking score and the orientation of the compounds in the active site compared with the obtained crystal structure.

A suite of computational and fast experimental methods identified four chloroacetamide warhead-based hits that covalently inhibit M^{pro} with K_{I} values of 11.5, 5.97, and 5.27 μM for compounds A, B, and C, respectively (K_{I} could not be determined for compound D). Binding modes—including covalent interactions—were elucidated via room temperature X-ray crystallography. The ligand-induced conformational changes characterized with MD suggest that the protein dynamics may be an important factor to consider in follow-up work to further improve affinity, thereby effectively lowering KI and reducing toxicity. Computational modeling and experimental validation of these four compounds using kinetic assays and MS characterization further revealed compound C as the most potent compound bound to M^{pro}. From the X-ray crystallographic studies, it was confirmed that the compound C forms a covalent interaction with the Cys145 and H-bond interactions with Leu141, Ser144, and His164 residues in the binding pocket of M^{pro} (FIG. 22). The oxygen atom of amide group of the compound C binds in oxyanion hole and its C3-phenyl group interacts with the hydrophobic Met165 residue from S4 subsite flanked by the side chain of Met49 from S2 subsite. We also observed a C—H . . . π interaction of the same C3-phenyl group with the His41 residue of the S2 subsite. As a result, Compound C forms stable interactions with chain A and its predicted binding pose orienting more closely to its crystal structure than in the case of the other chloroacetamides compounds.

An initial antiviral assay of the four studied chloroacetamide hits revealed toxicity in vitro that presents a challenge.45,46 However, chloroacetamide derivatives are most widely known for their use in herbicides, but they have recently demonstrated broad therapeutic potential including antimicrobial and anticancer properties of chloroacetamide derivatives. As such, it is cautioned against classifying the validated M^{pro} inhibitors as potential leads at this stage. Rather, the compounds serve as proof of principle that AI-assisted workflows can be leveraged for the rapid discovery of targeted covalent ligands. In an effort to advance the ability to account for toxicity and off-target effects in silico, follow-on work has been begun to optimize leads with graph neural networks and reinforcement learning approaches. In addition, the pipeline will incorporate a more robust toxicity assessment in the computational down-selection process to better tailor results to compounds with oral therapeutic potential.

From the MD simulations, RMSD and RMSF analyses reveal that binding of the ligand in the active site induces conformational changes in the active site region and in the dimer's interface of the M^{pro}. Also, the PCA motions and DCCM analysis demonstrate the collective conformational changes in the protein upon ligand binding. Most importantly, the simulations and extensive analysis identify allosteric changes. Moreover, interdomain motions indicate that the fluctuations in the D3 of chains A and B are due to binding of the ligand in the active site, which is similar to the observations found by the previous non-covalent study. The results suggest that the protein dynamics may be important factors to consider in follow-up work to further improve selectivity, thereby effectively lowering KD and reducing toxicity. Further integration of emerging techniques such as native MS with computational methods will complement the classical structural methods like crystallography for better definition of protein dynamics for structure-based drug design. This automated work can be applied to other systems to help advance the discovery of selective and potent covalent leads targeting other residues and protein targets.

### Example 41 Example M^{pro} Implementation: Materials and Methods—Covalent Inhibitor Design Workflow

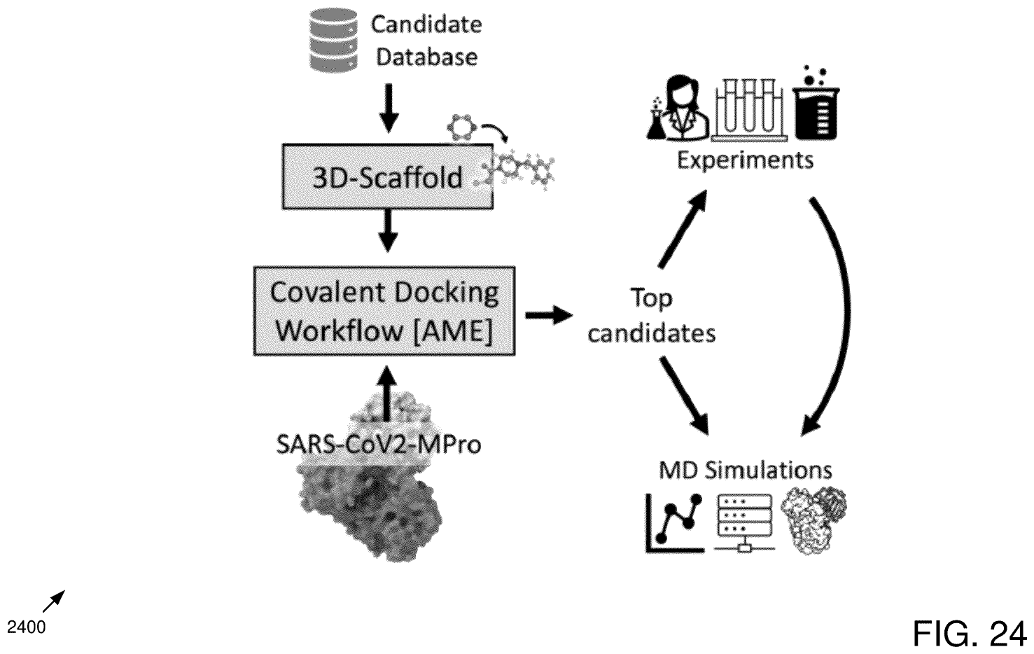

An Automated Modeling Engine for Covalent Docking was developed using AutoDockFR docking. The pipeline contains the components to automate covalent docking of specified electrophilic warheads to a CYS sidechain in the active site of the protein target (FIG. 18). Given a prepared receptor and a list of SMILES, the pipeline forms a covalent bond between the CYS sidechain and the appropriate functional groups. Aligning the attached CYS with the prepared receptor, the simulation then determines the energetically favorable poses of the ligand. The workflow (FIG. 24) utilizes RDKit and reaction SMARTS to prepare, predict, and rank binding poses for a set of input molecules. The automated workflow takes a prepared receptor, which defines the binding site, and a list of ligands in SMILES format. Each ligand is searched for substructures with defined thiol addition reactions and produces molecules in PDB format with an appropriately attached cysteine sidechain. The prepared ligand is then passed to AutoDockFR, which places the ligand at the binding site and searches the conformational space. AutoDockFR places the ligand by superimposing the added cysteine sidechain with the equivalent atoms of the receptor and utilizes a genetic algorithm to explore favorable binding site and conformations. The automation of the receptor preparation step is still under development. The entire workflow is available (https://github.com/nkkchem/AMEnCovDock).

Ligands were sorted according to their AutoDock4 score in kcal/mol. Scores are a function of various interaction forces between ligand-ligand and ligand-protein atom pairs, including van der Waals, electrostatic, hydrogen bonds, and desolvation contributions. The absolute values of docking score are presented, so a higher score implies tight binding between the ligand and receptor. For each molecule, all individually calculated pose values are averaged. The receptor was kept rigid during docking. Compound C docked the strongest out of the four compounds with a score of 8.72, tied with D. Compounds A and B had scores of 7.46 and 6.71 respectively. The drastic change between the scores of B and D due to a single addition of an isobutyl group is due to it stabilizing the protein in the pocket, holding the ligand's chlorine closer to His41. Compound C is instead stabilized by its oxygen through attraction to the Asn142 above the pocket.

FIG. 24 shows a computational covalent docking workflow for high-throughput virtual warheads screening against Cys145 of M^{pro}. It includes the DL model (3D-Scaffold) to generate warheads/scaffolds-based candidates, followed by AME for covalent docking of the generated candidates with M^{pro} to identify hit/potential candidates to be tested experimentally. Molecular Dynamics (MD) simulations are included.

### Example 42 Example M^{pro} Implementation: 3D-Scaffold Deep Learning Model

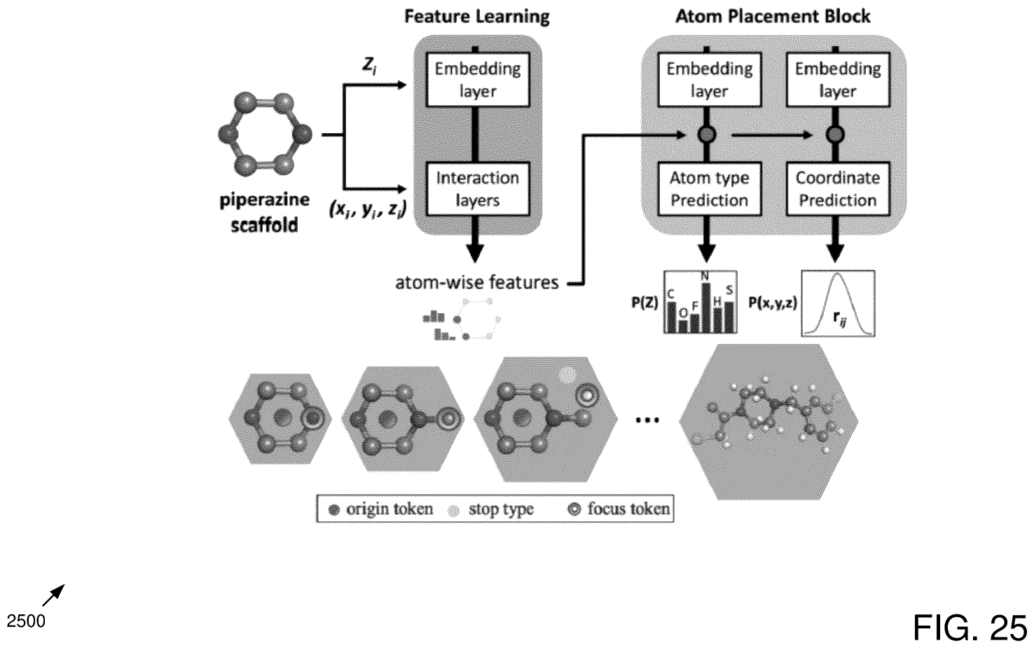

A deep tensor neural network-based deep generative model named 3D-Scaffold was developed. It generates three-dimensional coordinates of the molecules starting from given scaffold while preserving it. Input to the ML model are the 3D coordinates of the molecules in the training set and the SMILES strings of the corresponding scaffolds. 3D-Scaffold is an autoregressive model that generates the complete set of valid molecules for a given scaffold, adding atoms one at a time in 3D space. The model is trained end-to-end to learn the prediction of atom type to add to the scaffold and its corresponding distance from the given atom. For this, one can make use of 2 arbitrary atom types called tokens, namely origin token and focus token. The 3D-scaffold model considers the center of mass of the input scaffold as origin token and initialize molecule generation with focus token predicting the atom type and XYZ coordinates of the next atom. The process continues until valid molecule(s) is produced (FIG. 25). The general workflow for generating molecules from the 3D-Scaffold framework is shown in FIG. 25. Joshi et al., 2021 contains further details of the 3D-Scaffold framework. FIG. 25 shows that a 3D-scaffold DL framework was used as a generative model to produce covalent candidates with desired warhead and scaffold. In the example, a piperazine scaffold is iteratively extended by placing one atom after another and utilizing origin tokens, focus tokens, and stop type to aid molecular generation from the core. Warheads were instead used for the scaffold component.

To train the ML model, 5000 molecules from the FDA and Enamine databases were used. The goal was to train the ML model on the chemical space of druglike molecules and inverse design novel therapeutic candidates while building on desired scaffolds. The scaffold-based approach not only constrains the huge chemical space for inverse designing novel molecules to some local space of scaffolds, but preserving scaffolds also facilitates desired interaction with the target. Prior work has shown that compounds generated from the model typically have high binding affinity with the targets due to scaffolds which results in increased potency while designing a pharmaceutically relevant small drug molecule. Training datasets consist of 6 unique scaffolds, namely chloramides, acetamides, nitriles, pyrodines, acrylamides, maleamides. About 1000 molecules were generated from the model with piperazine and chloroacetamide as scaffolds. Generated molecules were examined for validity, uniqueness, and novelty. The molecules are then screened based on the synthetic accessibility (SA) score, quantitative estimation of drug-likeness (QED), and the partition coefficient (logP) properties before being used for subsequent docking evaluations. The molecules were further examined for desired binding activity against the target protein with docking and using MD simulations to narrow down top candidates.

### Example 42—Example M^{pro} Implementation: Molecular Dynamics (MD) Simulations

The crystal structure of SARS-CoV-2 3CL M^{pro} (PDB ID: 6WQF) was reported as an initial structure of the simulation. A cysteine residue at position 145 (Cys145) is replaced with covalent inhibitors using Discovery Studio Visualizer 19. GROMACS 2018.6 was used for all-atom molecular dynamics (MD) simulations of M^{pro} dimer with ligand bound in its chain A. The initial poses for covalent inhibitors were taken from the poses with the highest scores from our docking workflow. For the MD simulations, Amber FF14SB force field was adopted for the complex and TIP3P water model with Joung and Cheatham ion parameters were used for water and neutralizing monovalent ions in the system. To calculate partial charges of the covalent inhibitors and Cys145, the restrained electrostatic potential atomic partial charge (RESP) method at HF/6-31G* level was employed using NWChem 6.8.1. Simulations were performed in a cubic box composed of TIP3P water molecules and monovalent ions for neutralization of the system. The initial system was minimized by the conjugated gradient algorithms up to a maximum residual force of 10.0 KJ/mol. Then, the system was equilibrated at 300K for 500 ps under NVT ensemble using the Berendsen velocity rescaling method followed by 500 ps with NPT ensemble using the Berendsen pressure coupling method. Harmonic restraints with force constant of 1,000 KJ/mol were applied on the protein and substrate during equilibration steps. After the equilibrations, we performed production runs for 100 ns at 300 K and 1 atm using Parrinello-Rahman pressure coupling method while all the restraints were released at this step. The timestep adopted in all simulations is 2 fs and the particle mesh Ewald algorithm was used to evaluate long-range electrostatic interactions. The RMSD was analyzed using the GROMACS gmx rms module and RMSF using the GROMACS gmx rmsf module, and GROMACS covar and anaeig modules for calculating PCA and DCCM.

### Example 43—Example M^{pro} Implementation: Biochemical Characterization Determination of Inhibitor IC₅₀

For each compound, IC₅₀ curves were generated using a continuous fluorescence assay. Reaction volumes of 40 μL were used in Corning half area 96-well plates. First, 10 μL of purified M^{pro} (1 μM) in assay buffer (20 mM Tris-HCl, pH 7.35 with TCEP and EDTA) was added to the 10 μL of the appropriate inhibitor dilution in 20 mM Tris-HCl, pH 7.35 with 1.25% DMSO (typically 62.5, 15.625, 3.906, 0.977, 0.244, 0.061, 0.015, 0 μM in assay buffer) in each well and centrifuged briefly at 1000 g. 20 μL of Dabcyl-KTSAVLQSGFRKME-EDANS fluorescence peptide substrate (60 μM in 20 mM Tris-HCl, pH 7.35) was added to each well and the reaction progress was immediately monitored using a Biotek SynergyH1 plate reader at excitation 340 nm and emission 490 nm for 10 minutes at room temperature. The reactions had final concentrations of 250 nM enzyme, 30 μM peptide substrate, 100 μM TCEP, 0.5 mM EDTA, 2.5% DMSO in 20 mM Tris-HCl, pH 7.35. All the reactions were done in triplicate. For IC₅₀ determination, the initial rate data was used, and the kinetic values were obtained directly from nonlinear regression of substrate-velocity curves in the presence of various concentrations of the inhibitor. The equation Y=(Top-Bottom)/(1+1\hat{0}(X-log IC₅₀))+Bottom; X=log(concentration) and Y=binding; was used in the nonlinear regression.

### Example 44—Example M^{pro} Implementation: Determination of k_{inact} and K_{I}

Similar fluorescence assay was used to determine the time dependent IC50 curves. The plate with 10 μL of enzyme (1 μM) and 10 μL of the appropriate inhibitor dilution in 20 mM Tris-HCl, pH 7.35 with 1.25% DMSO was incubated at room temperature on a titer plate shaker with varying enzyme pre-incubation times (2, 15, 30, 45, 60 and 120 min) and centrifuged briefly at 1000 g just before incubation time ends. 20 μL of Dabcyl-KTSAVLQSGFRKME-EDANS fluorescence peptide substrate (80 μM in 20 mM Tris-HCl, pH 7.35) was added each well and the reaction progress was immediately monitored at excitation 340 nm and emission 490 nm for 10 minutes at room temperature using a plate reader ((Biotek Synergy H1). The reactions had final concentrations of 250 nM enzyme, 30 μM peptide substrate, 100 μM TCEP, 0.5 mM EDTA, 2.5% DMSO in 20 mM Tris-HCl, pH 7.35. All the reactions were done in triplicate. For IC₅₀ determination, the initial rate data was used, and the kinetic values were obtained directly from nonlinear regression of substrate-velocity curves in the presence of various concentrations of the inhibitor. The equation Y=(Top-Bottom)/(1+1\hat{0}(X-log IC₅₀))+Bottom; X=log(concentration) and Y=binding; was used in the nonlinear regression.

### Example 45—Example M^{pro} Implementation: Kₘ Determination

20 μL of enzyme (0.25 μM) in assay buffer (100 μM TCEP, 0.5 mM EDTA in 20 mM Tris-HCl, pH 7.35) was added to the 20 μL of Dabcyl-KTSAVLQSGFRKME-EDANS fluorescence peptide substrate with varying concentrations (0, 0625, 1.25, 2.5, 5, 10, 20, 40, 80, 160 μM in 20 mM Tris-HCl, pH 7.35) in each well. The reactions were immediately placed in a plate reader (Biotek SynergyH1) and the reaction progress was monitored at excitation 340 nm and emission 490 nm for 10 minutes. Kₘ was determined using Michaelis-Menten kinetics.

### Example 46—Example M^{pro} Implementation: M^{pro} Expression and Purification

Protein purification supplies were purchased from Cytiva (Piscataway, New Jersey, USA). The M^{pro} (NSP5) gene from SARS-CoV-2 was cloned into a construct in plasmid pD451-SR33 (Atum, Newark, CA) and expressed and purified as described by Kneller et al. (2020). In short, native termini are achieved by the NSP4-NSP5 autoprocessing sequence upstream of M^{pro} while downstream encodes the human rhinovirus 3C (HRV-3C) cleavage site followed by a His6-tag. Authentic N-terminal sequence is created by autocleavage during expression while the C-terminus is generated by HRV-3C treatment following Ni immobilized metal affinity chromatography.

### Example 47—Example M^{pro} Implementation: Protein-Inhibitor Co-Crystallization

Crystallization reagents were purchased from Hampton Research (Aliso Viejo, California, USA). Crystallographic tools were purchased from MiTeGen (Ithaca, New York, USA) and Vitrocom (Mountain Lakes, New Jersey, USA). Sitting-drop vapor diffusion was used to crystallize M^{pro} concentrated to ˜5.0 mg/mL in 20 mM Tris pH 8.0, 150 mM NaCl, 1 mM TCEP. Conditions for growing crystalline aggregates of ligand-free M^{pro} suitable for generating microseeds were identified by high-throughput screen at the Hauptman-Woodward Research Institute. Lyophilized samples of compounds A, C, and D (Enamine, Monmouth Jct., NJ, USA) were dissolved in 100% DMSO to make 50 mM stocks and stored at −20° C. Each compound was mixed with M^{pro} at 5:1 molar ratio and allowed to incubate at room-temperature for a minimum of one hour before solution clarification by centrifugation using 0.2 mm centrifugal filters. Single plate crystals suitable for room-temperature X-ray diffraction grew after 7 days at 14° C. in 28 μL drops of protein at a 1:1 mixture with 17-22% PEG3350, 0.1 M Bis-Tris pH 6.5 nucleated with 0.2 μL of microseeds at 1:200 dilution.

### Example 48 Example M^{pro} Implementation: Room-temperature X-Ray Data Collection and Structure Refinement

Protein crystals were mounted on loops encased in a MiTeGen (Ithaca, NY) room-temperature capillary setup. X-rays were generated from a Rigaku HighFlux HomeLab with a MicroMax-007 HF X-ray generator with Osmic VariMax optics. Diffraction images were collected using a DECTRIS Eiger R 4M hybrid photon counting detector. Crystallographic datasets were reduced and scaled using Rigaku CrysAlis Pro software. The structures were solved by molecular replacement using the inhibitor-free room-temperature M^{pro} structure (PDB ID 6WQF33) using Phaser Structure refinement was performed stepwise using phenix.refine from Phenix suite and manual refinement in COOT supported by Molprobity. Data collection and refinement statistics are listed in Table S3. The three room-temperature co-crystal complex structures and structure factors have been deposited into the Protein Data Bank with PDB accession codes 8DL9 for Mpro-A, 8DLB for Mpro-C, and 8DMD for Mpro-D complexes.

### Example 49—Example M^{pro} Implementation (Mass Spectrometry)

50 μL of 0.7 mg/mL (20 μM) M^{pro} stock solution was buffer exchanged twice into 100 mM ammonium acetate pH 7.5 with Zeba spin 7k MWCO spin column (ThermoFisher Scientific) for native MS analysis. For denaturing MS, ˜30 uL of 20 μM doubly exchanged M^{pro} and diluted to 500 μL with millipore H2O and concentrated with Amicon 3k MWCO spin filter (Millipore) to ˜40 μL for further desalting. For the reaction after buffer exchange, 20 μM compound was incubated with 5 μM protein at room temperature for 30 min, and then placed on ice before MS analysis. For denaturing MS, sample was diluted to 2 μM protein in 50% acetonitrile with 0.1% formic acid. For native MS, sample was diluted to 2 μM protein with 100 mM ammonium acetate pH 7.5.

Mass spectra were acquired on a Thermo Scientific Orbitrap Exploris 480. For denatured samples, the instrument was set to low pressure intact protein mode with the source fragmentation at 35 V, RF 70%, and a resolution of 120k at m/z 200. Under native conditions the Exploris instrument was set to high pressure intact protein mode with the source fragmentation between 75-135 V, (to reduce non-volatile salt adduction) and a resolution setting of 30k at 200 m/z. Raw mass spectra were deconvoluted into mass distribution using UniDec v4.4.1. “Smooth nearby points” and “Suppress artifacts” were selected as “Some”. Integrated peak areas were used to calculate the percentages of different species. For native MS data, the potassium adducts were grouped together by using the double deconvolution function with the control sample as “kernel”, which simplified the spectra.

### Example 50 Example M^{pro} Implementation: Cytotoxicity and Cytopathic Effect Assay

Evaluation of antiviral activity of compounds A-D was carried out in a 384-well white-bottom plate format as described in Bocci et al. Vero E6 (CRL-1586) cells were maintained using cell Minimum Essential Media with Earle's salts (EMEM, Corning Life Sciences) with 1% penicillin-streptomycin (P/S), 5% heat-inactivated fetal bovine serum (FBS) in 37° C. with 5% CO2. Briefly, on the day of infection, 5×10³ Vero E6 cells were seeded in 20 μL cell culture medium (containing 5% FBS) in 384-well plates, using a MultiFlo FX multi-mode microplate dispenser (BioTek). The same day, compounds were added to the cells (25 μL/well, in EMEM with 5% FBS, 1% P/S), with 2 wells per concentration. After 2 h of compound preincubation, 5 μL of virus was added to the wells using a multi-channel pipette to MOI=0.1. Viruses were diluted in EMEM with 5% FBS, 1% P/S and was kept on ice until cells were ready for infection. Eight virus-only control wells were included as the negative control and eight cells only were included as the positive control. The virus plus cells and the cells were used to normalize the data set. After 72 h, 30 μL of CellTiter-Glo (Promega) was dispensed into each well by a MultiFlo FX multi-mode microplate dispenser. After a 10 min incubation at room temperature, the plates were loaded into a Perkin EnVision multi-mode microplate reader to read luminescence.

### Example 50 Example Implementation: Further Details

Further details regarding the technologies can be found in McNaughton et al., “De novo design of protein target specific scaffold-based Inhibitors via Reinforcement Learning,” arXiv:2205.10473, MLDD workshop, which is hereby incorporated herein by reference along with supplemental materials.

Further details regarding the M^{pro} implementation can be found in Joshi et al., “AI-Accelerated Design of Targeted Covalent Inhibitors for SARSCoV-2,” J. Chem. Inf. Model. 2023, 63, 1438-1453, which is hereby incorporated herein by reference along with supplemental materials.

### Example 51—Example Advantages

A number of advantages can be achieved via the technologies described herein. For example, because the modeling is done in a three-dimensional context with reference to a particular target protein, more accurate training can be achieved, leading to higher quality candidates as described herein.

### Example 52—Example Computing Systems

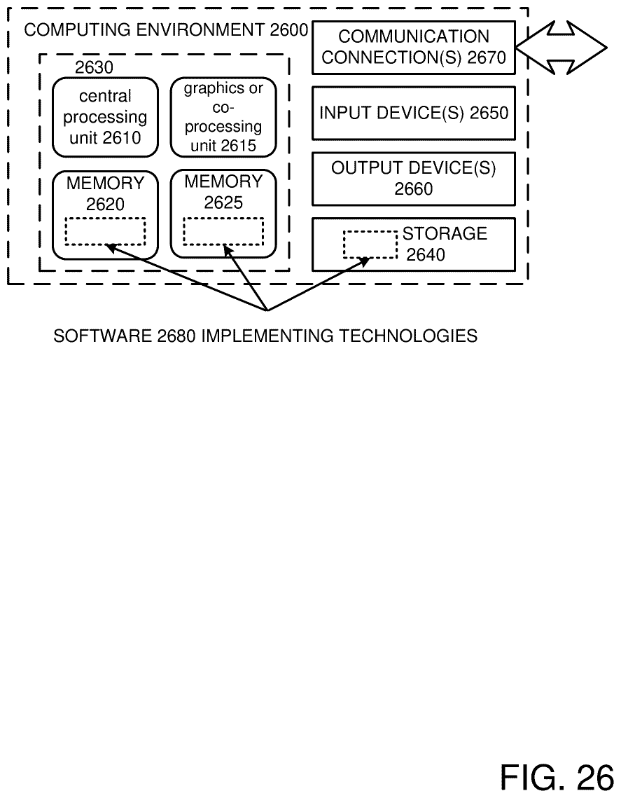

FIG. 26 depicts an example of a suitable computing system 2600 in which the described innovations can be implemented. The computing system 2600 is not intended to suggest any limitation as to scope of use or functionality of the present disclosure, as the innovations can be implemented in diverse computing systems.

With reference to FIG. 26, the computing system 2600 includes one or more processing units 2610, 2615 and memory 2620, 2625. In FIG. 26, this basic configuration 2630 is included within a dashed line. The processing units 2610, 2615 execute computer-executable instructions, such as for implementing the features described in the examples herein. A processing unit can be a general-purpose central processing unit (CPU), processor in an application-specific integrated circuit (ASIC), or any other type of processor. In a multi-processing system, multiple processing units execute computer-executable instructions to increase processing power. For example, FIG. 26 shows a central processing unit 2610 as well as a graphics processing unit or co-processing unit 2615. The tangible memory 2620, 2625 can be volatile memory (e.g., registers, cache, RAM), non-volatile memory (e.g., ROM, EEPROM, flash memory, etc.), or some combination of the two, accessible by the processing unit(s) 2610, 2615. The memory 2620, 2625 stores software 2680 implementing one or more innovations described herein, in the form of computer-executable instructions suitable for execution by the processing unit(s) 2610, 2615.

A computing system 2600 can have additional features. For example, the computing system 2600 includes storage 2640, one or more input devices 2650, one or more output devices 2660, and one or more communication connections 2670, including input devices, output devices, and communication connections for interacting with a user. An interconnection mechanism (not shown) such as a bus, controller, or network interconnects the components of the computing system 2600. Typically, operating system software (not shown) provides an operating environment for other software executing in the computing system 2600, and coordinates activities of the components of the computing system 2600.

The tangible storage 2640 can be removable or non-removable, and includes magnetic disks, magnetic tapes or cassettes, CD-ROMs, DVDs, or any other medium which can be used to store information in a non-transitory way and which can be accessed within the computing system 2600. The storage 2640 stores instructions for the software 2680 implementing one or more innovations described herein.

The input device(s) 2650 can be an input device such as a keyboard, mouse, pen, or trackball, a voice input device, a scanning device, touch device (e.g., touchpad, display, or the like) or another device that provides input to the computing system 2600. The output device(s) 2660 can be a display, printer, speaker, CD-writer, or another device that provides output from the computing system 2600.

The communication connection(s) 2670 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various embodiments. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer and should not be confused with acts performed by a human being. The actual computer operations corresponding to these terms vary depending on implementation.

### Example 53—Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing system to perform the method. The technologies described herein can be implemented in a variety of programming languages.

### Example 54—Example Cloud Computing Environment

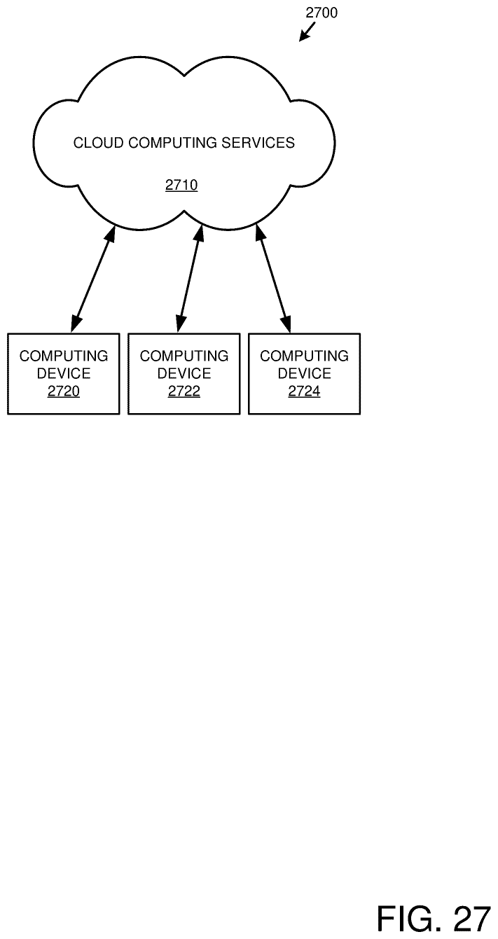

FIG. 27 depicts an example cloud computing environment 2700 in which the described technologies can be implemented, including, e.g., the system 100 of FIG. 1 and other systems herein. The cloud computing environment 2700 comprises cloud computing services 2710. The cloud computing services 2710 can comprise various types of cloud computing resources, such as computer servers, data storage repositories, networking resources, etc. The cloud computing services 2710 can be centrally located (e.g., provided by a data center of a business or organization) or distributed (e.g., provided by various computing resources located at different locations, such as different data centers and/or located in different cities or countries).

The cloud computing services 2710 are utilized by various types of computing devices (e.g., client computing devices), such as computing devices 2720, 2722, and 2724. For example, the computing devices (e.g., 2720, 2722, and 2724) can be computers (e.g., desktop or laptop computers), mobile devices (e.g., tablet computers or smart phones), or other types of computing devices. For example, the computing devices (e.g., 2720, 2722, and 2724) can utilize the cloud computing services 2710 to perform computing operations (e.g., data processing, data storage, and the like).

In practice, cloud-based, on-premises-based, or hybrid scenarios can be supported.

### Example 55—Example Implementations

Although the operations of some of the disclosed methods are described in a particular, sequential order for convenient presentation, such manner of description encompasses rearrangement, unless a particular ordering is required by specific language set forth herein. For example, operations described sequentially can in some cases be rearranged or performed concurrently.

### Example 56—Example Alternatives

The technologies from any example can be combined with the technologies described in any one or more of the other examples. In view of the many possible embodiments to which the principles of the disclosed technology can be applied, it should be recognized that the illustrated embodiments are examples of the disclosed technology and should not be taken as a limitation on the scope of the disclosed technology. Rather, the scope of the disclosed technology includes what is covered by the scope and spirit of the following claims.

What is claimed is:

1. A computing system comprising:
one or more processors;
memory;
an internal three-dimensional representation of an input target protein;
a generative machine learning model comprising an agent and a critic function; and
a training environment configured to execute a plurality of training episodes, wherein for a given training episode, the training environment performs (a)-(c):
(a) receiving a three-dimensional representation of a molecular scaffold as an internal three-dimensional representation of a molecule under construction;
(b) with an agent in a generative machine learning model, iteratively adding a representation of one or more atoms to the internal three-dimensional representation of the molecule under construction; and
(c) with a critic function in the generative machine learning model, calculating a reward, wherein calculating the reward comprises evaluating one or more binding criteria for the molecule under construction and the input target protein via the internal three-dimensional representation of the molecule under construction and the internal three-dimensional representation of the input target protein in a three- dimensional context;
wherein the training environment is further configured to perform training the generative machine learning model with the reward.

2. The computing system of claim 1, further comprising:
upon reaching a stopping condition, outputting the internal three-dimensional representation of the molecule under construction as a drug candidate for the input target protein.

3. The computing system of claim 1, wherein:
the critic function comprises parallel graph neural networks configured to determine binding probability indicating whether the molecule under construction binds with the input target protein.

4. The computing system of claim 1, wherein:
the agent comprises a three-dimensional molecule representation framework comprising a neural network implementing feature learning and atom placement.

5. The computing system of claim 4, wherein:
embedding and interaction layers are used to extract and update rotationally and translationally invariant atom-wise features that capture chemical environment of the molecule under construction; and
the features are used to predict distributions for a type of next atom and its three-dimensional coordinates.

6. The computing system of claim 1, wherein:
training the generative machine learning model with the reward comprises calculating gradients and tuning weights of the generative machine learning model.

7. The computing system of claim 1, wherein:
the binding criteria comprises a binding probability or a binding affinity.

8. The computing system of claim 1, wherein:
the reward comprises a compound reward comprising the one or more binding criteria and synthetic accessibility of the molecule under construction.

9. The computing system of claim 1, wherein:
the agent is configured to perform atom placement.

10. The computing system of claim 1, wherein:
the agent is configured to choose a type of the one or more atoms based on reward provided by the critic function.

11. The computing system of claim 10, wherein:
the agent is configured to choose a type of the one or more atoms based on probability
P_{Θ}(sₜ)^{type}=−log(\hat{p}_{type}^{Z}^{ₙₑₓₜ}).

12. The computing system of claim 1, wherein:
the agent is configured to add a representation of the one or more atoms to the internal three-dimensional representation of the molecule under construction at a distance from an already-placed atom represented in the internal three-dimensional representation of the molecule under construction, wherein the distance is based on a probability assigned by a three-dimensional-scaffold model.

13. The computing system of claim 12, wherein:
the agent is configured to choose the distance based on probability
$${{P}_{Θ} ( {s}_{t} )}^{dist} = {\sum }_{j = 1}^{N} {\sum }_{b ∈ B} {q}_{j}^{b} log ( {\hat{p}}_{j}^{b} ) .$$

14. The computing system of claim 1, wherein:
the critic function is configured to assess the molecule under construction a plurality of times for the given training episode.

15. The computing system of claim 1, wherein:
the agent and the critic function are configured to operate for a plurality of input target proteins.

16. A computer-implemented method comprising:
receiving a three-dimensional representation of a molecular scaffold as an internal three-dimensional representation of a molecule under construction;
with an agent in a generative machine learning model, iteratively adding a representation of one or more atoms to the internal three-dimensional representation of the molecule under construction, wherein the agent in the generative machine learning model is trained with a reinforcement learning process comprising rewards provided by a critic function evaluating one or more binding criteria of a plurality of training molecules under construction represented by internal three-dimensional representations of the training molecules under construction with an input target protein represented by an internal three-dimensional representation of the input target protein in a three-dimensional context;
reaching a stopping point; and
outputting the internal three-dimensional representation of the molecule under construction.

17. The method of claim 16, wherein:
the agent is configured to choose an atom type based on a probability distribution.

18. The method of claim 16, further comprising:
treating a subject with a molecule having a structure represented by the internal three-dimensional representation of the molecule under construction.

19. One or more non-transitory computer-readable media comprising computer-executable instructions that, when executed by a computing system, cause the computing system to perform operations comprising:
receiving a target-protein-agnostic generative machine learning model comprising an agent and a critic;
receiving an internal three-dimensional representation of a target protein;
training the target-protein-agnostic generative machine learning model with reinforcement learning, wherein the training comprises performing (a)-(c) for a plurality of training episodes:
(a) receiving a three-dimensional representation of a molecular scaffold as an internal three-dimensional representation of a molecule under construction;
(b) with an agent in a generative machine learning model, iteratively adding a representation of one or more atoms to the internal three-dimensional representation of the molecule under construction; and
(c) with a critic function in the generative machine learning model, calculating a reward, wherein calculating the reward comprises evaluating one or more molecular properties comprising one or more binding criteria for the molecule under construction and the target protein via the internal three-dimensional representation of the molecule under construction and the internal three-dimensional representation of the target protein in a three-dimensional context;
(d) communicating the reward to the agent, and modifying the agent according to the reward;
whereby the target-protein-agnostic generative machine learning model is trained as a target-protein-specific generative machine learning model operable to generate three-dimensional representations of molecules for binding to the target protein.

20. The one or more non-transitory computer-readable media of claim 19 further comprising computer-executable instructions that, when executed by a computing system, cause the computing system to perform operations comprising:
after the training, outputting the generative machine learning model as a trained model;
with the trained model, predicting a candidate molecule that modulates activity of the target protein in vivo.
